(19) United States
(12) Patent Application Publication
(10) Pub. No.: US 2025/0302142 A1
(43) Pub. Date: Oct. 2, 2025

(54) ESTIMATING METRICS FROM SENSORS OF A WEARABLE ARTICLE

(71) Applicant: NIKE, Inc., Beaverton, OR (US)

(72) Inventors: Haoyi T. Chen, Portland, OR (US); Michael Mazzoleni, Portland, OR (US); Marielle L. Morris, Portland, OR (US); Colin Arrowsmith, Verdun, CA (US); Ren Gibbons, Oakland, CA (US); Christopher Green, Austin, TX (US); Carmen Lai, Brisbane, TX (US)

(21) Appl. No.: 19/036,792

(22) Filed: Jan. 24, 2025

Related U.S. Application Data
(60) Provisional application No. 63/625,814, filed on Jan. 26, 2024.

(51) Int. Cl. A43B 3/44; A61B 5/00; A61B 5/103; A61B 5/11

(52) U.S. Cl. CPC A43B 3/44; A61B 5/1038; A61B 5/112; A61B 5/1123; A61B 5/6807; A61B 5/7264; A61B 2562/0219

(57) ABSTRACT

Aspects herein are directed to a estimating, among other things, changes in force representative of a force curve, human gait metrics, activity types, and/or other movement data from one or more sensors included in a wearable article. For example, at a first time, first sensor data via one or more sensors included in an article of footwear, is received. At a second time subsequent to the first time, second sensor data is received via the one or more sensors. Based at least in part on the first sensor data and the second sensor data, a change in force representative of at least a partial force curve is estimated. At least partially in response to the estimating, the received first sensor data, the received second sensor data, and/or a visual associated with the estimated change in force is sent, over a computer network and to a user device for presentation.

### CROSS-REFERENCE TO RELATED APPLICATIONS

This application claims priority to U.S. Provisional Patent Application No. 63/625,814 entitled “Estimating Metrics from Sensors of a Wearable Article,” filed Jan. 26, 2024, which is incorporated by reference in its entirety.

### FIELD

Aspects herein relate to systems and methods for estimating, among other things, changes in force representative of a force curve, human gait metrics, activity types, and/or other movement data from one or more sensors included in a wearable article, such as an article of footwear.

### BACKGROUND

Some technologies are designed to track various aspects of a user's health, fitness, movement, or other attributes. They use a combination of sensors and algorithms to monitor user activities, provide real-time data, and can sync with services or mobile apps for a more comprehensive experience. For example, wearable devices may synchronize with a cloud service to monitor a wearer's heart rate, track the wearer's daily activity, or record sleep patterns. However, these and other existing technologies are static in design and functionality, and unnecessarily consume computing resources, such as computer memory.

### BRIEF DESCRIPTION OF THE DRAWINGS

The present technology is described in detail herein with reference to the accompanying drawings, which are described below.

FIG. 1 illustrates, at a high level, a screenshot representing effects of a runner's feet experiencing a force over a threshold, according to some aspects.

FIG. 2 illustrates, at a high level, a screenshot representing effects of a gamer's feet experiencing a force over a threshold, according to some aspects.

FIG. 3A illustrates an article of footwear in a first position at a first time, according to some aspects.

FIG. 3B illustrates the article of footwear of FIG. 3A in a second position at a second time subsequent to the first time, according to some aspects.

FIG. 4 illustrates a screenshot of an example user interface, according to some aspects.

FIG. 5A illustrates an exploded view of an article of footwear with an embedded Inertial Measurement Unit (IMU), according to some aspects.

FIG. 5B illustrates a cross-sectional view of the IMU and midsole of the article of footwear of FIG. 5A, according to some aspects.

FIG. 6 illustrates a block diagram of a system, according to some aspects.

FIG. 7 is a schematic diagram illustrating how a neural network generates a force curve prediction, according to some aspects.

FIG. 8 illustrates an example Convolutional Neural Network (CNN), according to some aspects.

FIG. 9 is a schematic diagram illustrating how different layers of the same model can be split up on different devices to reduce storage consumption among other compute savings, according to some aspects.

FIG. 10 is a schematic diagram illustrating, at a high level, a force curve estimation or change in force estimation based on processing IMU data, according to some aspects.

FIG. 11 is a flow diagram of an example process for estimating a change in force over at least one parameter, according to some aspects.

FIG. 12 is a flow diagram of an example process for estimating one or more gait metrics, according to some aspects.

FIG. 13 is a flow diagram of an example process for estimating an activity type of a wearer associated with a wearable article, according to some aspects.

FIG. 14 is a flow diagram of an example process for classifying a gesture of a wearer, according to some aspects.

FIG. 15 is a flow diagram of an example process for extracting and providing three channels of sensor data as input to a model for estimation, according to some aspects.

FIG. 16 is a flow diagram of an example process for making an estimation of one or more metrics based on encoding and decoding model data via different devices, according to some aspects.

FIG. 17 is an example process for manufacturing an article of footwear with an embedded IMU, according to some aspects.

FIG. 18 is a block diagram illustrating an example computing environment for estimating one or more metrics from one or more sensors in an article of footwear, according to some aspects.

FIG. 19 is a block diagram of the electronics of any wearable article described herein, according to some aspects.

FIG. 20 is a block diagram of a computing device, according to some aspects.

### DETAILED DESCRIPTION

### Overview

Some existing technologies track a user's health, fitness, movement or other attributes. For example, some smart watches are configured to use sensors to determine steps taken, distance traveled, calories burned, heart rate, sleep duration, and/or sleep quality. Other technologies, such as virtual reality-based technologies, employ Head-Mounted Displays (HMDs) that include motion sensors for tracking the position and movement of the user within the virtual environment. Gaming technologies may also track movement of the user. However, all of these technologies and others are computing resource intensive. These technologies rely on services for data synchronization, storage, and analysis. Data collected by a wearable device is regularly synced to a cloud-based platform, where it is processed, analyzed, and made accessible through an app to view historical trends and the like.

There are various computing resource problems with these and other technologies. For example, one issue is that computing latency is unnecessarily increased because of the network communication channels that need to be opened for communication between cloud services and wearable devices, and the constant stream of real-time sensor data that needs to be transmitted over a network. For example, a wearable article may include a network interface that transmits protocoled packets (e.g., TCP/IP packets) of streamed sensor data to and from servers in near-real time to analyze and send notifications related to the sensor data. This causes latency problems because there is significant delay experienced when the sensor data transmitted to and from cloud services. Similarly there is excessive jitter with these technologies. Jitter refers to the variation in the delay of received data packets. Network devices use streaming buffers to temporarily store and forward streams of data packets. When buffers become full or data is held for prioritization, which is common in these technologies, it introduces jitter as packets are released at different times.

Some technologies are also static in design and functionality. For example, with respect to design, biomechanical research technologies require using bulky equipment, such as a force plate treadmill or other lab equipment, to function properly. However, using this technology requires tedious setup and configuration (e.g., mounting and wiring complicated stereo sensors and force plates). Some technologies are also static in functionality, which can lead to accuracy and other issues. For example, some technologies use a one-size-fits all model or function to make movement predictions. Some technologies also only use sensor data as inputs to make predictions.

Aspects of the present disclosure provide one or more technical solutions to one or more of these technical problems, as described herein. At a high level, certain aspects herein are directed to a wearable article system and methods for estimating, among other things, changes in force representative of at least a partial force curve, human gait metrics, activity types, and/or other movement data from one or more sensors included in an a wearable article (e.g., an article of footwear).

Some aspects have the technical effect of reduced memory consumption and reduced input/output (I/O). For example, with respect to memory consumption, a storage device (e.g., flash memory) at a wearable article stores only a first portion of a fuller model (e.g., a Convolutional Neural Network (CNN)), where an intermediate layer (e.g., a bottleneck or flattening layer) produces an estimation that contains fewer data points (e.g., 4 floats) relative to a fuller version of the model with a dense layer that would require more data points (e.g., 128 floats). Accordingly, for example, some aspects terminate a model early, which means to stop a forward pass of the network at the intermediate layer before reaching the final dense layer to make condensed predictions. In this way, more force curves or other estimated metrics can be stored to memory on-chip or at the wearable article, thereby reducing memory consumption.

In another example, with respect to reduced I/O and memory consumption, some aspects process only a few selected channels or axes (e.g., 3-axis IMU data) of a full-channel (e.g., a 6-axis IMU data) device. For example, some aspects exclude analysis of mediolateral axis accelerometer sensor data and only analyze the pitch axis sensor data from a gyroscope sensor, to reduce processing IMU sensor data from 6 channels to 3, which provides significant memory savings and reduced I/O. This is unlike existing technologies that store and analyze all real-time streamed sensor data. Instead of analyzing a constant stream of all sensor data, aspects only analyze selected channels. This means that aspects are performing fewer reads and/or writes, which reduces wear on components, such as a read/write head.

In yet another example, with respect to reduced memory consumption and reduced I/O, some aspects estimate one or more metrics based on processing only a fixed window of sensor data (e.g., indicative of a gait phase of a full gait cycle), where other sensor data indicative of other portions of the full gait cycle are not processed for estimation. For example, only sensor data representative of a detected “touchdown” gait phase are used to make force curve predictions. This means that sensor data representative of other gait phases are not stored to memory or otherwise used to make predictions, thereby reducing memory consumption and reducing I/O (e.g., because a read/write head is moving less to access such other sensor data).

Some aspects also improve the static design of existing technologies. For example, various aspects exclude a force sensor (e.g., a force transducer or load cell), which is a device that directly measures the force applied to it. For instance, instead of using bulky equipment, such as force plate treadmill common in biomechanical research technologies, aspects incorporate one or more sensors (e.g., an Inertial Measurement Unit (IMU), accelerometer, and/or a gyroscope) into a wearable article (e.g., an article of footwear). Accordingly, no tedious setup and configuration (e.g., mounting and wiring complicated stereo sensors and force plates) are required.

Some aspects also have the technical effect of improved accuracy relative to existing technologies. For example, instead of using a one-size-fits all model or function to make movement predictions, as with existing technologies, some aspects use a piecewise model to make metric predictions. A piecewise model is a mathematical model that includes multiple functions or expressions defined over different intervals or “pieces” of the input domain. In other words, instead of representing a single function that applies uniformly across its entire domain (e.g., all sensor data values corresponding to walking, jogging, and running or all gait phases), a piecewise model is a combination of different functions, each of which applies to a specific range of input values (e.g., sensor values indicative of running only (and not walking) or a specific gait phase, and not others). For example, one piecewise model may be a machine learning model that represents a range of sensor values representing a user running and another piecewise model may be a model that represents another range of sensor values representing a user walking. This approach is particularly useful with respect to accuracy because the relationship between running and walking (or particular gait phases) is not uniform and changes under different conditions or circumstances.

Some aspects also have the technical effect of improved accuracy by using more than sensor data as input to make predictions. For examples, in some aspects, estimations of force or changes in force (representative of a force curve) can be based on estimating one or more gait metrics (e.g., a foot strike angle, a quantity of strides, speed, etc.), estimating/classifying an activity type (e.g., running or walking) a wearer is in, and/or using a piecewise model. A particular wearer's foot speed, for example, may be indicative of a particular force value. If for example, it is estimated that the wearer is walking (classification of the activity type) and the wearer's speed (gait metric) is below a threshold indicative of walking, then aspects adjusts weights or otherwise increment a force curve estimation towards lower force because of the walking activity type and the speed being below a threshold.

Similarly, some aspects also have the technical effect of increased accuracy based on adding one or more run features (e.g., particular gait metrics) to a model, such as a Convolutional Neural Network (CNN). Such run features can be added to the model in a variety of ways, such as at the flatten layer and/or with the sensor data input at the first convolutional layer.

Some aspects also have the technical effect of increased accuracy based on having a model generating just a “shape” of a force curve and/or scaling the shape out (e.g., via a separate regression model). A regression model, for example, scales the shape of a curve by fitting a mathematical function or equation to a set of data points. Other functions, such as smoothing can additionally or alternatively be used. Smoothing is a technique used to reduce noise and emphasize patterns or trends in the input data, often by estimating a smoother curve. Smoothing techniques are applied to help reveal the underlying structure or relationships in the data and can be considered a form of regression in some cases. Therefore regression or smoothing techniques, especially in machine learning, can be used to help mitigate overfitting. Overfitting occurs when a model learns to fit the training data too closely, capturing noise and outliers in the data, rather than general patterns. Smoothing and some regression methods introduce constraints or reduce the model's complexity to make it less likely to overfit.

Some aspects also have the technical effect of reduced computing latency. In some aspects, this is at least partially due processing and/or transmitting only a few selected channels or axes (e.g., 3-axis IMU data) of a full-channel device (e.g., a 6-axis IMU), as described above. Additionally or alternatively, in some instances, this is because most or all functionality is performed at a wearable article (e.g., via a CNN stored on-chip). In this way, for example, network communication channels do not need to be opened for communication between cloud services and wearable devices (as much or at all), and/or fewer streams of real-time sensor data have to be transmitted over a network. For example, in some examples, a wearable article does not have to transmit as many protocoled packets (e.g., TCP/IP packets) of streamed sensor data to and from servers in near-real time by transmitting 3 channels instead of 6 channels or because the wearable article does not have to establish a communication session at all with the servers. This reduces latency because there is less delay experienced when the sensor data transmitted to and from cloud services. Similarly there is reduced likelihood of jitter because buffers store and forward fewer streams of data packets.

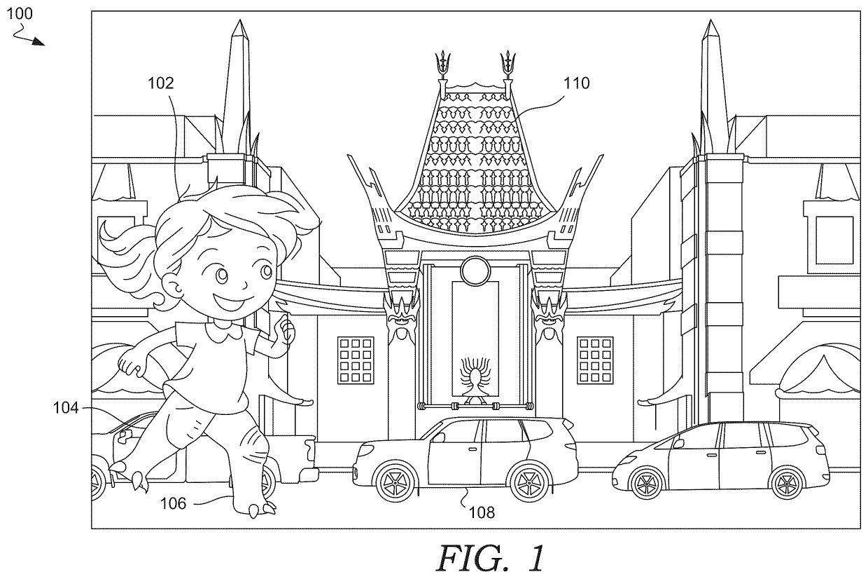

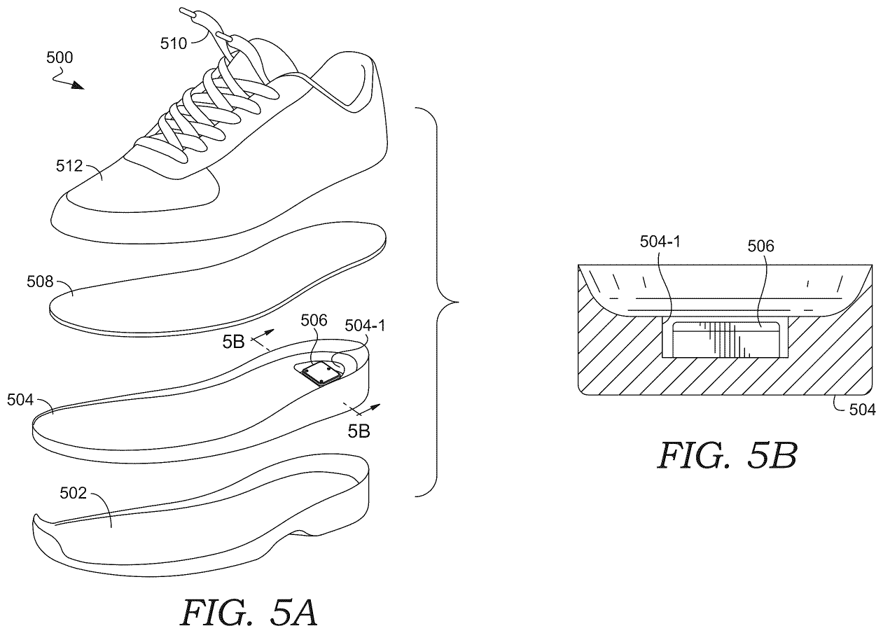

With respect now to the drawings, FIG. 1 illustrates, at a high level, a screenshot 100 representing effects of a runner's feet or corresponding sensor experiencing a force (e.g., Ground Reaction Force) over a threshold, according to some aspects. GRF, for example, is the force exerted by the ground on a body (e.g., a wearer's foot) in contact with it. Specifically, the screenshot 100 represents an Augmented Reality (AR), Virtual Reality (VR), or Mixed Reality (MR) environment where a real-world runner or user exceeds a force threshold over a threshold time period (e.g., 1 week), as measured by one or more real-world sensors (e.g., an accelerometer and/or gyroscope) incorporated into a real-world article of footwear (e.g., the article of footwear 500 of FIG. 5). In some aspects, the screenshot 100 represents a page of a computer application for performing gait analysis (e.g., for physical therapy or sports science) to understand the forces and mechanics involved in various movements and activities of a wearer. Alternatively or additionally, in some aspects, the screenshot 100 represents a game to reduce the force as much as possible to achieve the highest score.

In response to one or both of the force and/or time thresholds described above being met, some aspects generate or superimpose visual data 104 and 107 over the legs of the user's avatar 100, where such visual data 104 and 107 represents the avatar 100 growing legs of Godzilla. If the sensor readings fall under one or both thresholds, then the visual data 104 and 107 disappears such that visual data representing human legs are displayed instead of the visual data 104 and 107. Alternatively or additionally, in response to one or both thresholds being met, some aspects generate visual data (not shown) representing cracks across a display (e.g., the screenshot 100). Alternatively or additionally, in response to one or both thresholds being met and/or the avatar 100 coming within a threshold distance to the visual data 108 representing a car or the visual data 110 representing a building, some aspects change orientation of at least a portion of the visual data 108 or 110, which is indicative of deformation as a result of the Godzilla legs (i.e., the visual data 104 and 106) crushing the car or crashing the building. In some aspects, physics-based simulations can be used to represent such deformation. For example, collision detection algorithms can identify when data objects, like the car, collide with other objects or obstacles, such as the Godzilla legs. Alternatively or additionally, rigid body physics simulations may be used. These simulations model how objects respond to forces and collisions while maintaining their shape. Factors like mass, velocity, and the forces applied during collision may be considered.

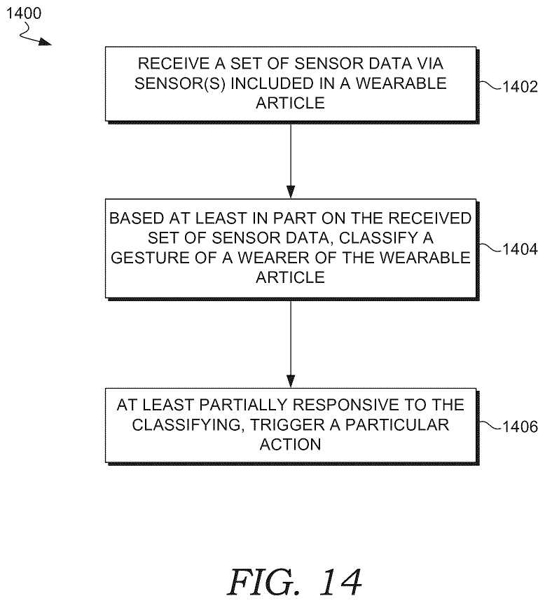

Although FIG. 1 illustrates what happens when a force threshold is met or exceeded, alternative or additional metrics can be measured and used to perform corresponding functionality. Other metrics include any of the gait metrics described herein. Examples of gait metrics include a quantity of strides a wearer makes, an activity type (e.g., running versus walking), foot strike angle, average peak, or the like. Accordingly, the force and/or time thresholds can be replaced with any gait metrics thresholds such that when any suitable threshold is met or exceeded (e.g., a foot strike angle), the visual 104/106, visual data representing cracks in the display, and/or crushing/crashing the car or building can be displayed, as described above. Other metrics may additionally or alternatively include gesture estimations, as described, for example with respect to FIG. 14. For example, in response to estimating a “stomp” gesture, the visual data 104 and/or 106 representing Godzilla's legs deform the visual data 108 representative of a crushed car.

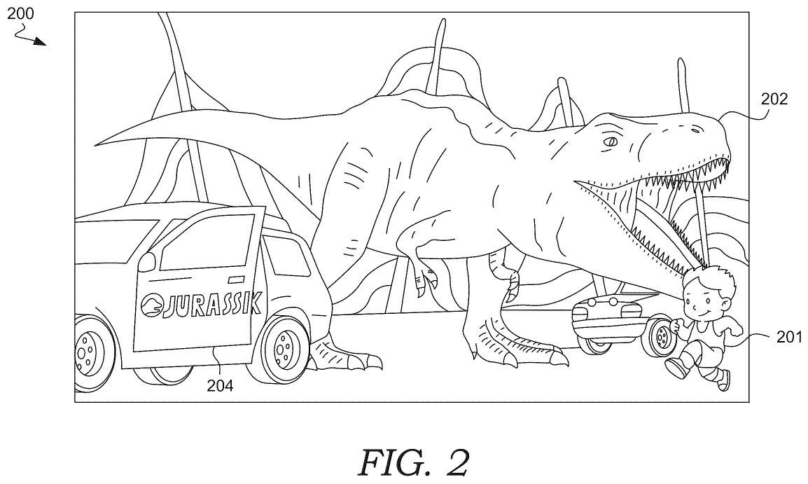

Turning now to FIG. 2, which illustrates, at a high level, a screenshot 200 representing effects of a gamer's feet experiencing a force (e.g., a GRF) over a threshold, according to some aspects. Specifically, the screenshot 200 represents a gaming environment where a user's article of footwear or corresponding sensor exceeds a force threshold, as measured by one or more sensors incorporated into an article of footwear (e.g., the article of footwear 500 of FIG. 5A). In some aspects, the screenshot 200 represents a page of a gaming application.

With respect to the screenshot 200, gamer avatar 201 needs to reach the car 204 within a time threshold (e.g., 10 seconds) while the real-world user has to run under a force threshold so that the T-Rex 202 is not alerted. In these aspects, the gamer avatar 200 represents a real-world user, which has on an article of footwear that includes one or more sensors (e.g., an IMU) and which measures or predict the force, where the avatar 201's movement mimics the user's real-world lower limb movements. In response to one or both of the force and/or time thresholds described above being exceeded (indicative that the user's fee had high impact and/or was too slow), the visual data 202 representing the T-rex changes position or orientation indicative of chasing or trying to grab the avatar 201. If the sensor readings fall under one or both thresholds (the user is running at low impact force within a given time), then the visual data 202 representing the T-rex stays in place or does not engaging in grasping the avatar 201. Although FIG. 2 illustrates what happens when a force threshold is met or exceeded, alternative or additional movement attributes can be measured and used to perform corresponding functionality, as described above, such as additional gait metrics, force curves, gait gestures, or the like.

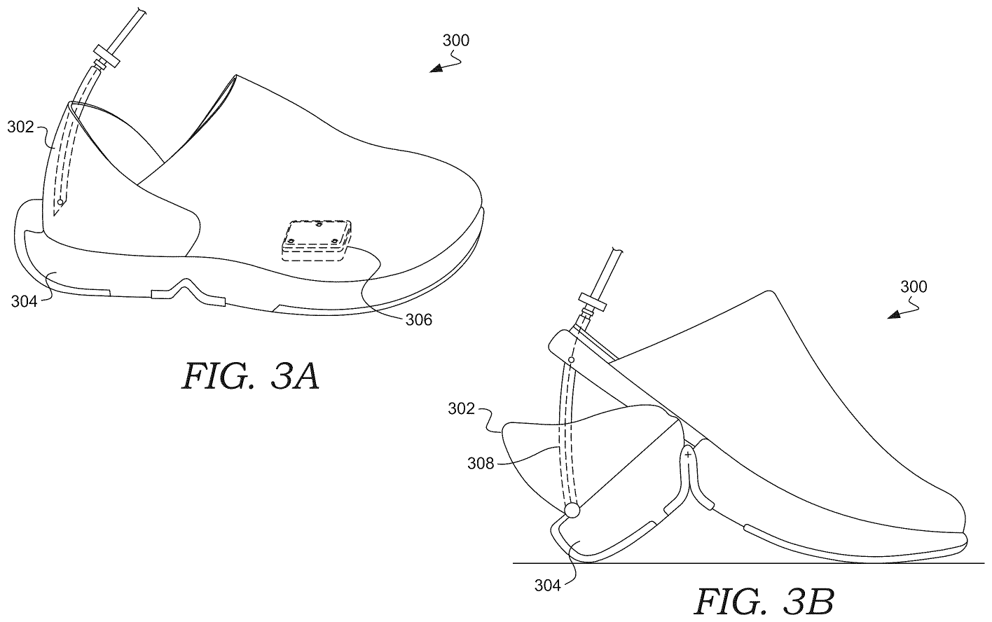

FIG. 3A illustrates an article of footwear 300 in a first position at a first time. The article of footwear 200 includes a heel counter 302, an outsole 304, and an IMU 306. FIG. 3B illustrates the article of footwear 300 of FIG. 3A in a second position at a second time subsequent to the first time. In response to the sensor data of the IMU 306 experiencing a force (e.g., a GRF) or other metric (e.g., gait metric) threshold, a spring mechanism 308 causes the outsole 304 to move along an axis closer to the ground and/or causes the heal counter 302 to move along an axis higher off the ground such that there is a “springing” force to resist or assist (e.g., cause higher magnitude of more of) the force. For example, particular aspects predict that the outer sole 304 at a toe portion of the article of footwear 300 is experiencing a GRF above a threshold at a metatarsal phalangeal portion, which is indicative of the wearer beginning to jump or run. Responsively, as illustrated in FIG. 3B, the spring 306 causes the springing force so as to assist the wearer in their jump or run. An example of the article of footwear 300 is described in PCT/US2023/031679, which is incorporated by reference herein in its entirety.

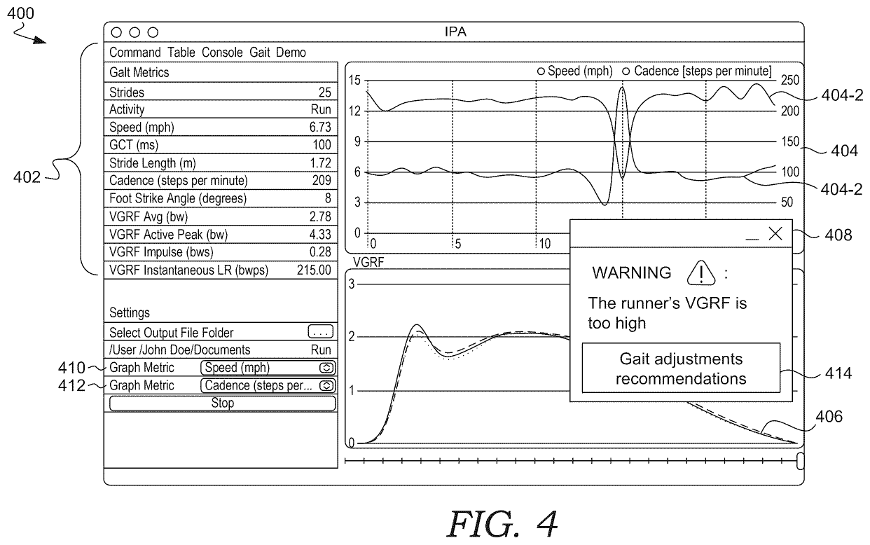

FIG. 4 illustrates a screenshot 400 of an example user interface, according to some aspects. In some aspects, the screenshot 400 represents a page of a coaching or other user application that warns a coach (or other user) when a gait metric or force exceeds a threshold. The screenshot 400 includes a set of gait metrics 402—i.e., strides (quantity of), activity type (run), speed (mph), Gait Cycle Time (in ms), stride length (in m), cadence (steps per minute), foot strike angle (in degrees), VGRF (Vertical Ground Reaction Force) average, VGRF peak, VGRF impulse, and VGRF instantaneous LR.

The screenshot 400 further includes a speed-cadence graph 404, and a GRF curve 406. The screenshot 400 further includes a popup window 404 indicating that the runner's VGRF is too high (i.e., it exceeds a threshold). The screenshot 400 further includes a gait adjustment recommendation button 414.

At a first time one or more sensors within an article of footwear may measure sensor data (e.g., accelerometer sensor data), which is then passed to and interpreted by one or more processors in order to derive the gait metrics 402, the speed-cadence graph 404, the popup window 408, and/or the force curve 406. In response to receiving an indication that a user has selected a “speed” graph metric as indicated in 410 and a “cadence” graph metric as indicated in 412, particular aspects automatically cause presentation of the speed-cadence graph 404, where the speed is indicated by the line 404-1 and the cadence is indicated by the line 404-2.

The vGRF curve 406 is a graphical representation of an estimation/prediction of how the vertical force changes as a person interacts with the ground while performing various movements. Such curve 406 may be used in biomechanics and gait analysis to understand the distribution and timing of forces during activities like walking, running, and jumping. The curve 406 can provide valuable insights into the mechanics and efficiency of these movements and may be useful for assessing aspects of gait and sports performance. In some aspects, the horizontal axis of the curve 406 represents time, with data points collected at regular intervals. This axis shows the duration of a specific activity or movement, such as walking, running, or jumping. In some aspects, the vertical axis of the curve 406 represents the magnitude of the vertical ground reaction force. In some aspects, this force is measured in units of Newtons (N) or another appropriate force unit. The force axis shows the variations in force applied to the ground by a person's foot or body during the activity over time.

Although the popup window 408 indicates that the runner's VGRF is too high, it is understood that VGRF is representative only, and that any movement or gait metric may be represented, such as the quantity of strides or speed falling outside of a threshold. In response to receiving an indication that the user has selected the “gait adjustment recommendations” button 414, particular aspects cause presentation of one or more recommendations that, if taken by the wearer, help reduce or eliminate a gait metric or other movement value falling outside of a threshold. For example, in response to receiving an indication that the user has selected the button 414, particular aspects access a data structure stored in memory, which lists predetermined hand-coded steps the user can take to reduce their VGRF. For instance, such data structure may be a lookup table, where the index or key of a particular entry or record represents a particular VGRF range and the value represents the action that the user must take. In an illustrative example, one key may be 2000 to 2,050 Newtons and the value might be, “tell the runner to run more on the balls of their feet and not the heel,” or the like. Accordingly, if the runner does take such action, this may eliminate the notification in the popup window 408.

FIG. 5A illustrates an exploded view of an article of footwear 500 with an embedded Inertial Measurement Unit (IMU) 506, according to some aspects. In some aspects, an article of footwear includes a sole structure secured to an upper. The article of footwear 500 described herein may comprise a shoe or any part of a shoe (e.g., a midsole, a sockliner, or an upper), a sock, a running shoe, a baseball shoe, a basketball shoe, a skateboarding shoe, a cycling shoe, an American football shoe, a tennis shoe, a global football shoe, a training shoe, a walking shoe, a hiking shoe, and the like. The concepts described herein may also be applied to other footwear types that are considered non-athletic such as dress shoes, loafers, sandals, and work boots. As used herein, the article of footwear may be divided into different general regions. A forefoot region generally includes portions of the article of footwear that correspond to the toes and joints connecting the metatarsals with the phalanges. A midfoot region generally includes portions of the article of footwear corresponding with an arch area and an instep area of the foot. A heel region generally corresponds with rear portions of the foot including the calcaneus bone. The article of footwear described herein may include a lateral side which corresponds with an outside area or lateral portion of the foot (i.e., the surface that faces away from the other foot) and a medial side which corresponds with an inside area of the foot (i.e., the surface that faces toward the other foot). The different regions and sides described above are intended to represent general areas of footwear to aid in the following discussion and are not intended to demarcate precise areas. The different regions and sides may be applied to the article of footwear as a whole, to the upper, and to the sole structure.

Continuing with FIG. 5A, the article of footwear 500 includes an outsole 502, a midsole 504, an insole 508, and an upper 512. The midsole 504 is configured to be joined between the insole 508 and the outsole 502. The midsole 504 includes a cavity or pocket 504-1, which is configured to receive an IMU 506. FIG. 5B illustrates a cross-sectional view of the IMU 506 and midsole 504 of the article of footwear of FIG. 5A, according to some aspects. Accordingly, FIG. 5B further illustrates the midsole 504, the cavity 504-1, and the IMU 506. In some aspects, the IMU 506 is equal to or greater than 21×17.5 mm (e.g., plus or minus 5%) but smaller than 2 inches by 2 inches (e.g., plus or minus 5%). Accordingly, such cavity 504-1 may be slightly larger than these dimensions (e.g., plus 5%) to fit the IMU 506. In some aspects, the midsole 504 can be made from any suitable material to accommodate the IMU 506, while providing cushioning and support. For example, in some aspects, the midsole 504 is or includes ethylene vinyl acetate (EVA) foam.

To manufacture the midsole 504, a mold for the midsole 504 can be created with the designed cavity 504-1 for the IMU 506. Particular aspects then inject or pour the chosen midsole material into the mold, ensuring that the cavity 504-1 for the IMU 506 is properly formed. Then aspects allow the midsole 504 to cure or set. Once the midsole 504 is ready, particular aspects embed the IMU 506 into the designated cavity 504-1. If the IMU 506 requires wiring or connections to other components (e.g., a battery or sensor), some aspects run the wiring through the midsole 504 to connect the IMU 506 to the necessary components. Some aspects seal the midsole 504 to protect the IMU 506 from moisture, impact, and other potential sources of damage. For example, sealing can include filling in the cavity 504-1 with additional EVA foam such that the IMU 506 is completely encompassed or surrounded by the midsole 502 (e.g., there is no opening for the IMU 506 to fall out of). This can be done using a sealant or cover that is also integrated into the midsole 504-1. Once the midsole 504 is ready, it can be incorporated into the rest of the shoe during the shoe manufacturing process, following the typical steps for stitching and assembly. For example the midsole 504 can be joined on top of the outsole 502 and the insole 508 can be joined to the midsole 504, and the like in any suitable manner. To “join” as described herein means to stitch, fasten, or otherwise couple two articles together. For example, joining can include or mean to sew, knit, weave, bond, join in a non-woven manner, or the like.

Continuing with FIG. 5A and FIG. 5B, the IMU 506 can be any suitable IMU with any suitable quantity and types of sensors. In some aspects, the IMU 506 is configured to detect or measure the rotational movement of 6 channels, such as Pitch, Roll, and Yaw of a gyroscope and longitudinal, vertical, and mediolateral of an accelerometer. To achieve such, the IMU 506 may incorporate the functionality of one or more accelerometers, gyroscopes, and/or magnetometers. Accelerometers may serve as the tool for velocity measurement on the IMU 506. In some aspects, the accelerometer's functionality is to detect the rate of change in velocity of an object (e.g., the article of footwear 500). In some aspects, Gyroscopes serve as the tool for rotation/rotational rate measurement on the IMU 506. Its functionality is to detect rotational changes or maintain orientation. Magnetometer sensors measure strength, and/or direction of a magnetic field on an IMU. Combining these three sensors gives measures orientation, velocity, and magnetic field force.

It is understood that while the IMU 506 is illustrated in FIG. 5A and FIG. 5B as being embedded within the midsole 504, it can be embedded or coupled to other components of the article of footwear 500. For example, in some aspects it is embedded within the insole 508, embedded within the outsole 502 (as described with respect to the midsole 504), attached to the laces 510, or otherwise fixed to the upper 512. In another example, the IMU 506 can be embedded in a sockliner, as described in US provisional application 63/465,969, filed on May 12, 2023, which is incorporated by reference in its entirety. In yet another example, the IMU 506 can be embedded in the article of footwear 500, as described in US application U.S. Pat. No. 7,607,243, which is incorporated by reference in its entirety.

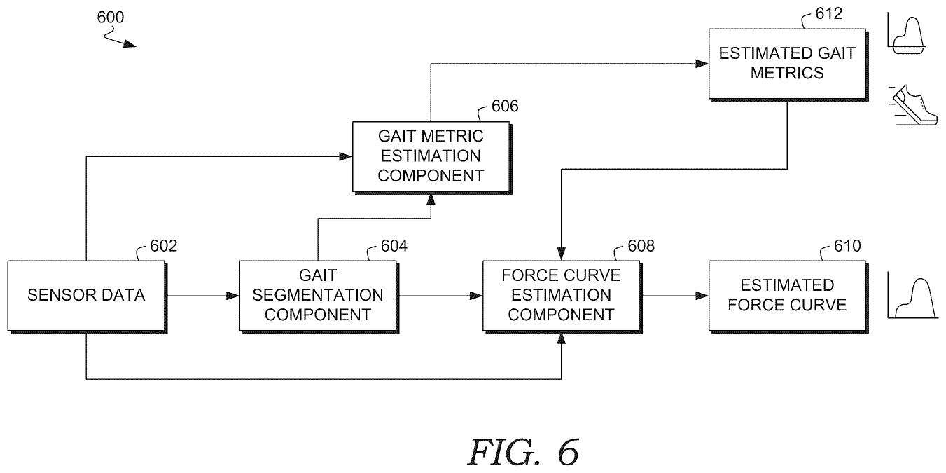

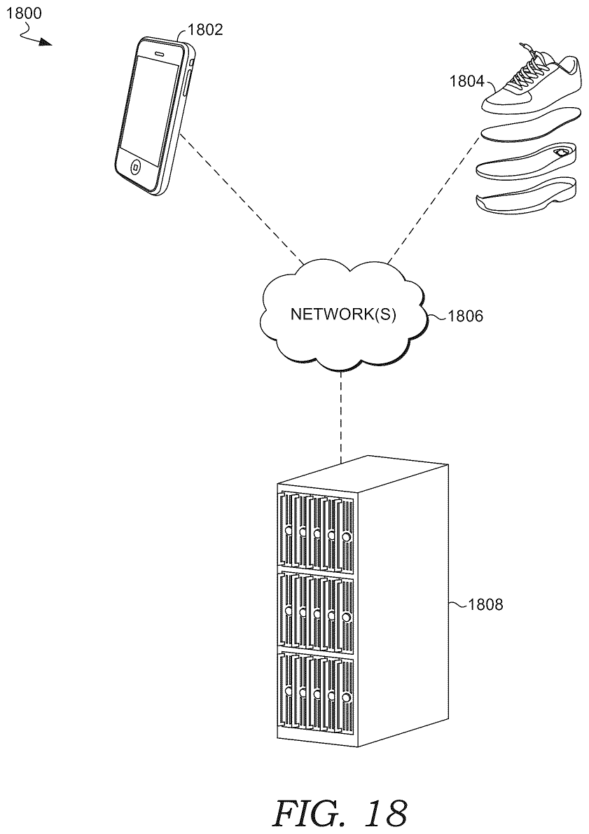

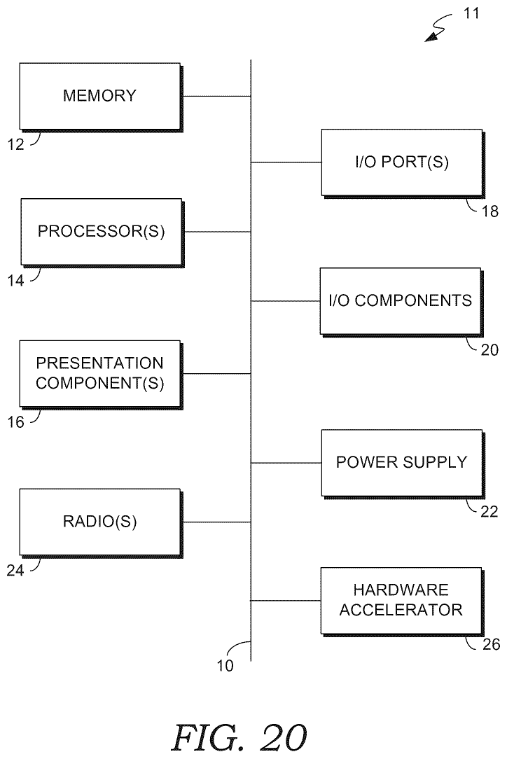

FIG. 6 illustrates a block diagram of a system 600, according to some aspects. The system 600 represents only one example of a suitable computing system architecture. Other arrangements and elements can be used in addition to or instead of those shown, and some elements may be omitted altogether for the sake of clarity. Further, many of the elements described herein are functional entities that may be implemented as discrete or distributed components or in conjunction with other components, and in any suitable combination and location. For example, some or each of the components of the system may be located within a single computing device (e.g., the computing device 8 of FIG. 20). Alternatively, some or each of the components may be distributed among various computing devices, such as in a distributed cloud computing environment. In some embodiments, the system 600 and each of the components are located within the article of footwear 1804, system 1808, and/or the user device 1802, as described in more detail in FIG. 18. In some aspects, the components of the system 600 represent or are embodied as a set of compiled computer instructions or functions, program modules, computer software services, logic gates, hardware accelerators, or an arrangement of processes carried out on one or more computer systems.

The system 200 generally operates to estimate a force curve and estimate gait metrics. The system 200 includes sensor data (e.g., IMU sensor data transmitted from the IMU 506 of FIG. 5A), a gait metrics estimation component 606, a gait segmentation component 604, a force curve estimation component 608, estimated gait metrics 612, and estimated force curve 610. At a first time the sensor data 602 is transmitted to the gait metrics estimation component 606, the gait segmentation component 604, and/or the force curve estimation component 608.

The gait segmentation component 604 is generally responsible for detecting, estimating, classifying, predicting, or otherwise computing which activity type a wearer is in and/or deriving (e.g., programmatically calling) the appropriate corresponding model. Examples of activity types are running, walking, jogging, or gait phases of a gait cycle. The gait cycle, which is a sequence of events that occur during one complete cycle of walking, running, or the like, is typically divided into several gait phases. Initial Contact (IC), for example, is the moment when the foot first makes contact with the ground, typically with the heel. Loading Response (LR) phase is when the foot begins to bear weight, and the body starts to move over the supporting limb. The foot continues to pronate (roll inwards) during this phase. Midstance (MSt) is when the body's weight is directly over the supporting limb. The foot is flat on the ground, and the arch begins to rise. Terminal Stance (TSt) is when the body continues to move forward over the supporting limb. The heel starts to lift, and the foot supinates (rolls outward). Pre-Swing (PSw) is when the foot lifts off the ground, and the body begins to prepare for the swing phase. Initial Swing (ISw) is the beginning of the leg's forward swing as it leaves the ground. In Mid-Swing (MSw), the leg continues to swing forward in this phase. Terminal Swing (TSw) is when the leg prepares to make contact with the ground in this final phase of the swing.

In an illustrative example of how the gait segmentation component 604 functions in some aspects, at a first time the gait segmentation component 604 classifies a particular stride, and/or a particular window of sensor data values from the sensor data 602 as a particular activity type, such as running or walking. In some aspects, such classification includes the concept of using a machine learning model that has been trained to learn relationships between sensor data values and corresponding labels or classifications, such as walking or running. For example, in some aspects, during training or fine-tuning, a model is fed a range of sensor data values that are labeled as “running.” Additionally or alternatively, the model is fed a range of other sensor data values that are labeled as “walking.” Accordingly, over multiple epochs, the model learns to reduce its error and adjust its weights to learn which sensor data values are indicative of walking or running. For instance, because running is higher impact than walking, running may be associated with a higher range of accelerometer values than walking. Accordingly, a machine learning model may learn such relationship. Accordingly, at model deployment time or inference time, the model can make an inference (e.g., that a wearer is in a running phase) according to particular received sensor data values based on training on the same or similar sensor data values. Responsive to such classification of activity type, some aspects programmatically call a piecewise model that represents or is associated with the force curve estimation component 608, as described in more detail below.

The gait metric estimation component 606 is generally responsible for detecting, estimating, classifying, predicting, or otherwise computing one or more gait metrics of a wearer (e.g., of the article of footwear 500). The output of the gait metric estimation component 606 is the estimated gait metrics 612. Examples of gait metrics include strides (quantity of), activity type (run), speed (mph), Gait Cycle Time (in ms), stride length (in m), cadence (steps per minute), foot strike angle (in degrees), VGRF (Vertical Ground Reaction Force) average, VGRF peak, VGRF impulse, and VGRF instantaneous LR. The gait metric estimation component 606 takes, as input, the sensor data 602 and/or the output of the gait segmentation component 604 (e.g., a classification that the wearer is running), each of which is described in more detail below.

In an illustrative example of how the gait metric estimation component 606 functions in some aspects, at a first time the gait metric estimation component 606 extracts a particular window of sensor data values from the sensor data 602 and/or an activity type from the gait segmentation component 604 and responsively estimates a particular gait metric (e.g., Vertical Ground Reaction Force—VGRF) and corresponding value. In some aspects, such classification includes the concept of using a machine learning model (e.g., a regression model) that has been trained to learn relationships between sensor data values and/or activity type classifications (from gait segmentation component 604) and corresponding labels or classifications, such as a particular predicted quantity of force (though other gait metrics may be made). For example, in some aspects, during training or fine-tuning, a model is fed a range of sensor data values (e.g., accelerometer and gyroscope values X) and/or outputs from the gait segmentation component 604 (e.g., “walking”) that are labeled as “force between a range of 400 and 500 Newtons” (or other gait metric). Additionally or alternatively, the model is fed a range of other sensor data values (e.g., accelerometer and gyroscope values Y) and/or gait segmentation outputs (e.g., “running”) that are labeled as “force between a range of 700 and 800 Newtons.” Accordingly, over multiple epochs, the model learns to reduce its error and adjust its weights to learn which sensor data values and/or activity type classifications are indicative of a particular range of gait metrics, such as force, quantity of strides, foot strike angle, and/or the like. Accordingly, for example, at model deployment time or inference time, the model can make an inference (e.g., that a wearer is experiencing a particular VGRF value) according to particular received sensor data values based on training on the same or similar sensor data values.

In some aspects, the gait metric estimation component 606 uses additional or alternative algorithms. For example, an accelerometer may measure linear acceleration. It provides information about the forces acting on an object in a specific direction (e.g., in three axes-X, Y, and Z or longitudinal, vertical, and mediolateral). When a device (e.g., the IMU 506) is at rest on a flat surface, in some aspects, the accelerometer measures the acceleration due to gravity (approximately 9.81 m/s²) in the opposite direction of the gravity vector (upwards in the Z-axis). A gyroscope measures angular velocity, which is the rate of rotation or change in orientation. Gyroscopes provide information about the rate of rotation in each of the device's axes. Gyroscope data can be integrated over time to estimate the orientation or angular displacement of the device.

To predict force or acceleration using accelerometer and gyroscope data, some aspects use sensor fusion techniques, and/or with the help of a complementary filter (e.g., a Kalman filter), or a more advanced sensor fusion algorithm. By integrating the accelerometer data twice (in the absence of any other forces), some aspects obtain the change in position. When gravity is removed (by subtracting the gravity component from the accelerometer data), the remaining acceleration data can be used to calculate the position and/or orientation of the device (e.g., an accelerometer). The gyroscope data provides information about the rate of change in orientation. By integrating this data over time, various aspects estimate the orientation of the device with respect to an initial reference frame.

Combining the orientation data from the gyroscope with the linear acceleration data from the accelerometer allows aspects to calculate the position and/or orientation of the device (e.g., an IMU 506) in its current orientation. This fusion process compensates for the fact that accelerometers measure both linear acceleration and the constant force of gravity. The combined data can be used to estimate the position and/or orientation of the device in real-time. For example, if the device is moving, various aspects compute the net force responsible for that motion, taking into account its orientation and acceleration.

The force curve component 608 takes, as input, the sensor data 602, the estimated gait metrics 612, and/or the activity type classifications (e.g., generated from the gait segmentation component 604) to generate the estimated force curve 610 as an output. Because a force curve is a graphical representation of how a force changes over time or in relation to a specific event or activity (e.g., a particular gait phase, running, or walking), in some aspects, the output 610 simply represents a magnitude or indicator of force changes over time in relation to the specific event or activity (as opposed to a graphical representation). For example, the output 610 may simply represent estimated changes in force over at least one parameter, such as time and/or according to a particular activity type classification. The shape of the force curve describes how the force changes over time or during an event. Different patterns can indicate different characteristics of the activity. For example, in a running gait analysis, the force curve may show the pattern of forces as the foot strikes the ground, rolls through, and pushes off. Force curves often have specific features that correspond to key events or phases in an activity. For instance, in a vertical jump analysis, the curve will typically show a sharp increase in force leading up to take-off, then little to no force at take-off, then a subsequent a sharp increase again when landing. Researchers and/or users may use force curves to analyze various aspects, such as the timing and magnitude of peak forces, loading rates, force distribution between different body parts or limbs, and other biomechanical or physical properties. This analysis can offer insights into performance, injury risk, and the mechanics of an activity. Force curves are a useful tool for understanding the mechanics and kinetics of various movements and activities, and they help users make informed decisions about improving performance, preventing injuries, and optimizing equipment and techniques.

As described above, in some aspects, the gait segmentation component 604 programmatically calls another model for the force curve estimation component 608 to use, such as a piecewise model. A piecewise model, also known as a piecewise function or piecewise-defined function, is a mathematical model that includes multiple functions or expressions defined over different intervals or “pieces” of the input domain. In other words, instead of representing a single function that applies uniformly across its entire domain (e.g., all sensor data values corresponding to walking, jogging, and running or all gait phases), a piecewise model is a combination of different functions, each of which applies to a specific range of input values (e.g., sensor values indicative of running only (and not walking) or a specific gait phase, and not others). For example, one piecewise model may be a machine learning model that represents a range of sensor values representing a user running and another piecewise model may be a model that represents another range of sensor values representing a user walking. This approach is particularly useful because the relationship between running and walking (or particular gait phases) is not uniform and changes under different conditions or circumstances.

In an illustrative example, in response to the gait segmentation component 604 classifying a set of sensor values as “running,” the gait segmentation component 604 can return a “running” piecewise model to the force curve component 608 to use, which is directly used to make the estimated force curve 610. This helps with being able to more granularly estimate changes in force over time, as sensor data and thus force may be significantly different between running and walking, for example.

In some aspects, such piecewise model or prediction of the estimate force curve 610 includes the concept of using a machine learning model that has been trained to learn relationships between sensor data values, activity type classifications (from gait segmentation component 604), and/or the estimated gait metrics 612 and corresponding labels or classifications, such as a particular predicted change of force over time, which is described in more detail below. For example, in some aspects, during training or fine-tuning, a model is fed a range of sensor data values (e.g., accelerometer and gyroscope values X), gait metrics values (e.g., foot strike angle at time X), and/or a walking model from the gait segmentation component 604 that uses regression techniques to predict the shape of the force curve. Accordingly, over multiple epochs, the model learns to reduce its error and adjust its weights to learn which sensor data values, activity type, and/or gait metrics classifications are indicative of a particular force curve or its corresponding shape. Accordingly, for example, at model deployment time or inference time, the model can make an inference (e.g., that there is a particular change in force over time as represented by a force curve) based on training on the same or similar data.

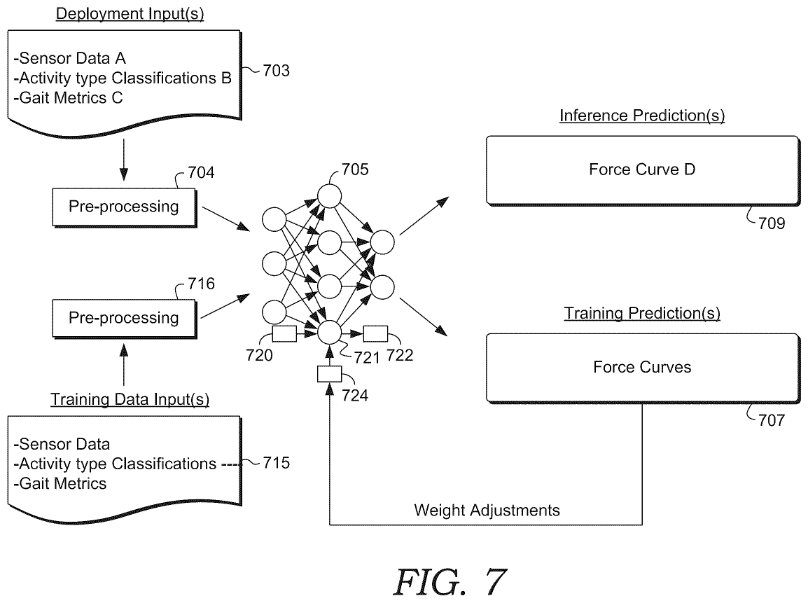

FIG. 7 is a schematic diagram illustrating how a neural network 705 generates a force curve (or change in force) prediction, according to some aspects. In some embodiments, the neural network 705 represents what is used by or included in the force curve estimation component 608 of FIG. 6. In some aspects the neural network 705 represents or includes any suitable model functionality, such as supervised learning (e.g., using logistic regression, using back propagation neural networks, using random forests, decision trees, etc.), unsupervised learning (e.g., using an Apriori algorithm, using K-means clustering), semi-supervised learning, reinforcement learning (e.g., using a Q-learning algorithm, using temporal difference learning), a regression algorithm (e.g., ordinary least squares, logistic regression, stepwise regression, multivariate adaptive regression splines, locally estimated scatterplot smoothing, etc.), an instance-based method (e.g., k-nearest neighbor, learning vector quantization, self-organizing map, etc.), a regularization method (e.g., ridge regression, least absolute shrinkage and selection operator, elastic net, etc.), a decision tree learning method (e.g., classification and regression tree, iterative dichotomiser 3, C4.5, chi-squared automatic interaction detection, decision stump, random forest, multivariate adaptive regression splines, gradient boosting machines, etc.), a Bayesian method (e.g., naïve Bayes, averaged one-dependence estimators, Bayesian belief network, etc.), a kernel method (e.g., a support vector machine, a radial basis function, a linear discriminate analysis, etc.), a clustering method (e.g., k-means clustering, expectation maximization, etc.), an associated rule learning algorithm (e.g., an Apriori algorithm, an Eclat algorithm, etc.), an artificial neural network model (e.g., a Perceptron method, a back-propagation method, a Hopfield network method, a self-organizing map method, a learning vector quantization method, etc.), a deep learning algorithm (e.g., a restricted Boltzmann machine, a deep belief network method, a convolution network method, a stacked auto-encoder method, etc.), a dimensionality reduction method (e.g., principal component analysis, partial lest squares regression, Sammon mapping, multidimensional scaling, projection pursuit, etc.), an ensemble method (e.g., boosting, bootstrapped aggregation, AdaBoost, stacked generalization, gradient boosting machine method, random forest method, etc.), and/or any suitable form of machine learning algorithm.

The neural network 705 is modeled as a data flow graph (DFG), where each node (e.g., 721) in the DFG is an operator with one or more input and output tensors, such as 720 and 722. A “tensor” (e.g., a vector) is a data structure that contains values representing the input, output, and/or transformations processed by the operator. Each edge of the DFG depicts the dependency between the operators. Neural network 705 includes an input layer, an output layer and one or more hidden layers. An Input layer is the first layer of the neural network 705. The input layer receives pre-processed (e.g., via the pre-processing 704 or 716) input data represented by 703 and 715, such as one or more sensor data values (e.g., particular accelerometer and gyroscope values). The Output layer is the last layer of neural network 705. The output layer generates one or more inferences in the form of clustering, regression, classifications, or the like, which can either be hard classification (e.g., a force curve has shape X) or soft probabilities (e.g., 50% likely that the force curve has shape X), which is represented by the predictions 709 and 707. Neural network 705 may include any number of hidden layers. Hidden layers are intermediate layers in neural network 705 that perform various operations.

Each node in FIG. 7, such as node 721, is associated with or includes one or more activation tensors, such as input tensor 720, output tensor 722, and/or intermediate tensors. An “activation tensor” is a tensor that is an input, intermediate, and/or output to at least one neural network layer (e.g., as modeled going from left to right), as illustrated by the flow of data from input tensor 720 to output tensor 722. This is different than a weight tensor, such as 724, where weight tensors are modeled as flowing upward (not being actual inputs or outputs). In other words activation tensors represent some form of the neural network inputs 703 and 715. For example, the input tensor 720 or node 721 can represent whether particular sensor data values inside or outside of a threshold were present or estimated, whereas a weight tensor represents the weight values indicating node activation/inhibition values.

Each node in the network 705 may also be associated with or include and/or one or more weight tensors (e.g., 724), which include weight values. A “weight” in the context of machine learning may represent the importance or significance of a feature or feature value for prediction. For example, each feature (e.g., accelerometer values within a range) may be associated with an integer or other real number where the higher the real number, the more significant the feature is for its prediction. In one or more aspects, a weight in a neural network represents the strength of a connection between nodes or neurons from one layer (an input) to the next layer (a hidden or output layer). A weight of 0 may mean that the input (e.g., the input tensor 720) will not change the output (e.g., the output tensor 722), whereas a weight higher than 0 changes the output. The higher the value of the input or the closer the value is to 1, the more the output will change or increase. Likewise, there can be negative weights. Negative weights may proportionately reduce the value of the output. For instance, the more the value of the input increases, the more the value of the output decreases. Negative weights may contribute to negative scores. For example, force curve estimations may be highly correlated with a specific accelerometer value range, a specific gait metric range, and/or activity type, and so neural network layers or nodes representing these ranges of values and/or activity type may be weighted higher so that that this data is activated or taken into account when making a final prediction score.

Each node of the neural network 705 may additionally perform one or more functions using the activation tensors and weight tensors, such as activation functions, matrix multiplication, normalization, or the like. In some aspects, the nodes in the neural network 705 are fully connected or partially connected. Continuing with FIG. 7, each node may process one or more inputs in 703 and 715 (or portion thereof) using activation tensors and weight tensors. In some aspects, in response to receiving the deployment input(s) 703 and the training data input(s) 715, the neural network 705 first performs pre-processing 704 or 716, such as encoding or converting such input into machine-readable indicia representing the entire input (e.g., a tensor representing sensor data values over a particular time span). Responsively, the node may then receive an input tensor, which may, for example, represent whether one or more features (e.g., particular sensor value ranges) are present in the input. In some aspects, the input tensor is an N-dimensional tensor, where N can be greater than or equal to one. In some aspects, an input tensor 720 represents the input data of neural network 705 if the node is in the input layer. In some aspects, the input tensor 720 is also the output of another node in the preceding layer. In some aspects after a node, such as the node 721, performs an operation using the input tensor 720, it generates an output tensor 722, which is then passed to the other neurons in the hidden layer and/or output layer. The output tensor 722 represents the output processed by the node 721. For example, the output tensor 722 may be a matrix representing the product of matrix multiplication or a matrix indicating whether the sensor data values have exceeded a threshold (or are within a range). In various aspects, the output tensor 722 represents an input of another node in the succeeding layer (i.e., the output layer).

In some aspects, node 721 applies a weight tensor 724 to the input tensor 720 via a linear operation (e.g., matrix multiplication, addition, scaling, biasing, or convolution). All other nodes in the neural network may perform identical functionality. In some aspects, the result of the linear operation is processed by a non-linear activation, such as a step function, a sigmoid function, a hyperbolic tangent function (tan h), and rectified linear unit functions (ReLU) or the like. The result of the activation or other operation is an output tensor 722 that is sent to a subsequent connected node that is in the next layer of neural network 705. The subsequent node uses the output tensor 722 as the input activation tensor to another node.

Each of the functions in the neural network 705 may be associated with different coefficients (e.g., weights and kernel coefficients) that are adjustable during training. For example, after preprocessing 716 (e.g., normalization, feature scaling and extraction) in various aspects, the neural network 705 is trained using one or more data sets of the preprocessed training data inputs 715 in order to make acceptable loss training predictions at the appropriate weights to set the weight tensors. This will help later at deployment time to make correct inference predictions 709. In one or more aspects, learning or training includes minimizing a loss function between the target variable (for example, a prediction indicating an incorrect force curve) and the actual predicted variable (for example, a prediction indicating a correct force curve). Based on the loss determined by a loss function (for example, Mean Squared Error Loss (MSEL), cross-entropy loss, etc.), the loss function learns to reduce the error in prediction over multiple epochs or training sessions so that the neural network 705 learns which features and weights are indicative of the correct inferences, given the inputs. Accordingly, it is desirable to arrive as close to 100% confidence in a particular classification or inference as much as possible so as to reduce the prediction error. In an illustrative example, the neural network 705 learns that for a given set of sensor data values A, gait metrics values B, and a particular activity type (e.g., running), the correct classification is a particular force curve or predicted change of force over a particular time period.

Subsequent to a first round/epoch of training, the neural network 705 makes predictions with a particular weight value, which may or may not be at acceptable loss function levels. For example, the neural network 705 may process the pre-processed training data inputs 715 a second time to make another pass of predictions. This process may then be repeated over multiple iterations or epochs until the weight values in the weight tensors are learned for optimal or correct predicted values (for example, by maximizing rewards and minimizing losses) and/or the loss function reduces the error in prediction to acceptable levels of confidence.

In some aspects, before the training data input(s) 715 (or deployment input(s) 703) are provided as input into the neural network 705, the inputs are preprocessed at 716 (or 704). In some aspects, such pre-processing includes feature scaling, feature extraction, normalization, and the like. Scaling (or “feature scaling”) is the process of changing number values (e.g., via normalization or standardization) so that a model can better process information. For example, some aspects can bind number values between 0 and 1 via normalization. Other examples of preprocessing includes feature extraction, handling missing data, feature scaling, and feature selection.

Feature extraction involves computing a reduced set of values from a high-dimensional signal capable of summarizing most of the information contained in the signal. Feature extraction techniques develop a transformation of the input space onto the low-dimensional subspace that attempts to preserve the most relevant information. In feature selection, input dimensions that contain the most relevant information for solving a particular problem are selected. These methods aim to improve performance, such as estimated accuracy, visualization, and comprehensibility. An advantage of feature selection is that important information related to a single feature is not lost, but if a small set of features is required and original features are very diverse, there is chance of information being lost as some of the features must be omitted. On the other hand, with dimensionality reduction, also known as feature extraction, the size of the feature space can often be decreased without losing information about the original feature space.

In some aspects, the pre-processing of the data at 716 and/or 704 includes missing data techniques. In some aspects, these missing data techniques include complete case analysis, single imputation, log-linear models and estimation using the EM algorithm, propensity score matching, and multiple imputations. The technique confines attention to cases for which all variables are observed in a complete case analysis. In a single implicit imputation method, missing values are replaced by values from similar responding units in the sample. The similarity is determined by looking at variables observed for both respondent and non-respondent data. Multiple imputations replace each missing value with a vector of at least two imputed values from at least two draws. These draws typically come from stochastic imputation procedures. In the log linear model, cell counts of a contingency table are modeled directly. An assumption can be that, given expected values for each cell, the cell counts follow independent multivariate Poisson distributions. These are conditional on the total sample size, with the counts following a multinomial distribution.

In some aspects, the preprocessing at 716 and/or 704 includes outlier detection and correction techniques for handling outlier data within the input data 715/703. Outliers, by virtue of being different from other cases, usually exert a disproportionate influence on substantive conclusions regarding relationships among variables. An outlier can be defined as a data point that deviates markedly from other data points. For example, error outliers are data points that lie at a distance from other data points because they result from inaccuracies. More specifically, error outliers include outlying observations that are caused by not being part of the targeted population of data, lying outside the possible range of values, errors in observation, errors in recording, errors in preparing data, errors in computation, errors in coding, or errors in data manipulation. These error outliers can be handled by adjusting the data points to correct their values or more such data points from the data set. In some implementations, particular aspects define values more than three scaled median absolute deviations (“MAD”) away from the median as outliers. Once defined as an outlier, some aspects replace the values with threshold values used in outlier detection.

In some aspects, the preprocessing at 716 and/or 704 includes feature selection at the input data 715 and/or 703. Feature selection techniques can be performed for dimensionality reduction from the extracted features. The feature selection techniques can be used to reduce the computational cost of modeling, to achieve a better generalized, high-performance model that is simple and easy to understand. Feature extraction techniques can be performed to reduce the input data's dimensionality. However, in some implementations, the resulting number of features may still be higher than the number of pre-training data 715. Therefore, further reduction in the dimensionality of the data can be performed using feature selection techniques to identify relevant features for classification and regression. Feature selection techniques can reduce the computational cost of modeling, prevent the generation of a complex and over-fitted model with high generalization error, and generate a high-performance model that is simple and easy to understand. Some aspects use the mRmR sequential feature selection algorithm to perform feature selection. The mRmR method is designed to drop redundant features, which can design a compact and efficient machine learning-based model.

In one or more aspects, the neural network 705 converts or encodes the deployment input(s) 703 and training data input(s) 715 into corresponding feature vectors in feature space (for example, via a convolutional layer(s)). A “feature vector” (also referred to as a “vector”) as described herein may include one or more real numbers, such as a series of floating values or integers (for example, [0, 1, 0, 0]) that represent one or more other real numbers, a natural language (for example, English) word and/or other character sequence (for example, a symbol (for example, @, !, #), a phrase, and/or sentence, etc.). Such natural language words and/or character sequences correspond to the set of features and are encoded or converted into corresponding feature vectors so that computers can process the corresponding extracted features. For example, aspects can parse, tokenize, and encode each value or other content in pages into one or more feature vectors.

Continuing with FIG. 7, in some aspects, the neural network 705 is trained in a supervised manner using annotations or labels, which represent continuous numerical values based on using a regression model. At least one objective of a regression model is to predict or estimate a numerical value based on the input features. In some aspects, the evaluation of regression models involves metrics like Mean Squared Error (MSE), Mean Absolute Error (MAE), Root Mean Squared Error (RMSE), and R-squared (R²). These metrics measure the accuracy and precision of the model's numerical predictions.

In an illustrative example, in some aspects, training includes (or is preceded by) annotating/labeling training data 715 so that the neural network 705 learns associations between the features or weights and corresponding labels, which is used to change the weights/neural node connections for future predictions. For example, particular aspects capture or sample different sensor data ranges, gait metric ranges, and activity type classifications at a first time and then output that information as a report. Responsively, wearers, subject matter experts, or programming logic then label such report with a particular force value range (between X and Y Newtons) or force change (from time 0 to the first time, there is a change of Z Newtons of force). Such process can repeated for various subsequent times so that labels can be made for different time in order to, for example, provide near real-time predictions indicative of a force curve based on a sequence of time a wearer wears a wearable article. In this way, the neural network 705 can learn which weights or features and their corresponding sensor data, gait metric, and/or activity type values are indicative of a particular force or force curve. As such, the neural network 705 accordingly adjusts the weights (the weight tensors) or deactivates nodes such that certain nodes corresponding to certain sensor data, gait metric, and/or activity type values are activated and other nodes corresponding to other certain sensor data, gait metric, and/or activity type values at particular locations are inhibited to make score predictions.

In one or more aspects, subsequent to the neural network 705 training, the neural network 705 (for example, in a deployed state) receives one or more of the pre-processed deployment input(s) 703. When a machine learning model is deployed, it has typically been trained, tested, and packaged so that it can process data it has never processed. Responsively, in one or more aspects, the deployment input(s) 703 (i.e., the sensor data values A, the activity type classifications B, and the gait metrics C) (e.g., indicative of a particular time slice and/or activity that a wearable article is experiencing) are fed to the neural network 705, which then uses the same weight tensors (e.g., 724) that were learned via training so that the neural network 705 can produce the correct inference predictions 709. For example, the input tensor 720 can include new values (e.g., sensor data values indicated in 703), which is then multiplied or otherwise combined with the weight tensor 724, representing the same weight values learned at training, in order to make the inference prediction(s) 709.

In some aspects, the sensor data in the input(s) 703 and/or 715 represent the sensor data 602 of FIG. 6. In some aspects, the activity type classifications in the input(s) 703 and/or 715 represent the output produced by the gait segmentation component 604. In some aspects, the gait metrics of the input(s) 703 and/or 715 represent the estimated gait metrics 612 of FIG. 6 and as produced by the gait metric estimation component 606.

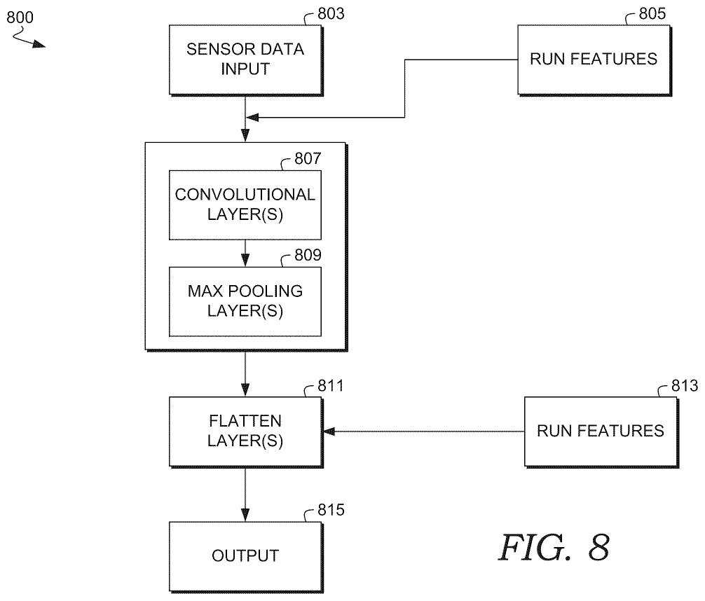

Referring now to FIG. 8, illustrates an example Convolutional Neural Network (CNN) 800, according to some aspects. In some aspects, the CNN 800 represents the neural network 705 of FIG. 7. The CNN 800 receives the sensor data (e.g., the sensor data 602) and the run features 805 and/or 813. The CNN 800 includes a convolutional layer(s) 807, a max pooling layer(s) 809, a flatten layer(s) 811, and the output 815.

In some aspects, the sensor data 803 represents any suitable sensor data described herein, such as the sensor data provided by the IMU 506 of FIG. 5A and FIG. 5B. In some aspects, the input data includes 6 channels, such as 3-axis accelerometer data (longitudinal, vertical, and mediolateral) and 3-axis gyroscope (pitch, yaw, and roll). Channels represent the different measurement or sensor data readings collected from various sensors. Alternatively, in some aspects, there are 3 channels-only the pitch axis for gyroscope data and the longitudinal and vertical accelerometer sensor data. In some aspects, the sensor data input 803 includes 256 samples. Samples, also known as “batch size” or “batch dimension,” represent the number of independent data points or examples that are fed into the CNN 800 in a single forward or backward pass. For example, there may be 256 sensor data readings fed into the CNN for a single forward pass.

In some aspects, the run features 805 are also fed into the CNN 800 as input to the convolutional layer 807. In some aspects, the run features 805 and/or 813 represent any of the gait metrics described herein, such as described with respect to the estimated gait metrics 612 of FIG. 6. In some aspects, the run features 805 have a shape of (256, 2). The input data shape refers to the dimensions of the data that is fed into the CNN 800. For example, the shape may specify the number of samples and the length of the time series at which sensor data is sampled.

The convolutional layer(s) 807 applies a set of learnable filters or kernels to the sensor data input 803 and/or run features 804 to extract features. Each filter (a small matrix) detects specific patterns or features. Convolution involves sliding the filter over the sensor data input 803 and/or the run features 805, element-wise multiplying and summing the values, and producing an output feature map. The output represents the presence of the detected features in different spatial locations. Convolutional layer(s) 807 may have parameters like the number of filters, filter size, and padding, which can be adjusted to control the CNN 800's capacity and receptive field. In some aspects, there are 4 filters with a kernel size of 12. The kernel size refers to the dimensions of the filter, specifically its width and height.

The max pooling layer(s) 809 is generally responsible for performing max pooling. Max pooling is a downsampling operation applied to the feature maps generated by convolutional layer(s) 807. The max pooling layer 809 reduces the spatial dimensions of the feature maps while preserving the most important information. Max pooling operates by dividing the feature map into non-overlapping regions (e.g., 2×2 or 3×3) and selecting the maximum value from each region to create a new, smaller feature map. This helps reduce the computational complexity, makes the CNN 800 more robust to variations in object position and size, and helps control overfitting. In some aspects, the pool size of the max pooling layer(s) 809 is 6. Pool size refers to the dimensions of the local region over which the max pooling operation is applied. The pool size is typically specified as a tuple with two values, (width, height), and it determines the size of the region over which the max operation is applied.

The flatten layer(s) 811 may be used to transition from the convolutional and pooling layers 807 and 809 respectively to the fully connected layers (not shown) of the CNN 800. For example, in some aspects, the flatten layer(s) 811 takes the 2D or 3D tensor output from the previous layers (e.g., the feature map) and converts it into a 1D vector. This flattening process may be useful because fully connected layers may require a 1D input. Once flattened, the vector can be fed into one or more fully connected layers to make final predictions at the output 815. In some aspects, the flatten layer(s) 811 also takes the run features 813 (e.g., shape of 2) as input to create the 1D vector. In some aspects, the run features 813 represent the exact same run features 805 but in some aspects, they are different run features. In some aspects, the run features 805 or 813 are introduced as input according to alternative embodiments, such as either with the sensor data input 803 to be processed by the convolutional layer(s) or alternatively at the flatten layer(s) 811. In some aspects, the run features 813 represent a very specific pool of gait metrics, which corresponds to a shape of 4.

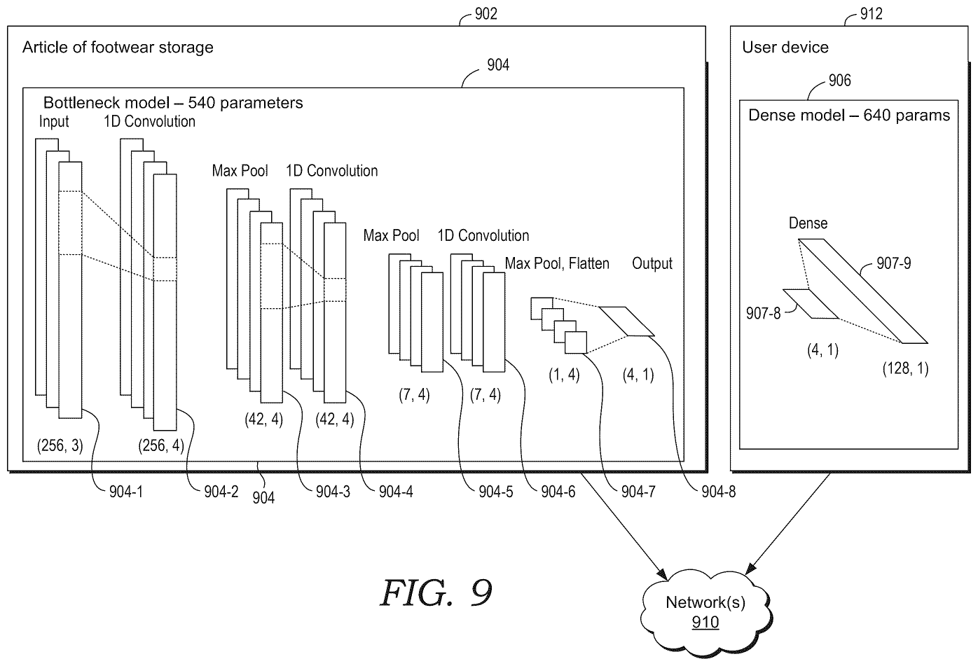

FIG. 9 is a schematic diagram illustrating how different layers of the same model can be split up on different devices to reduce storage consumption among other compute savings, according to some aspects. FIG. 9 includes an article of footwear storage 902 (e.g., RAM, cache, flash, or other memory), the network(s) 910 (e.g., a Wide Area Network (WAN)), and the user device 912. The article of footwear storage 902 includes the bottleneck model 904. The user device 912 includes the dense model 906.

In some aspects, the article of footwear storage 902 is embedded in or otherwise joined to a wearable article, such as the article of footwear 500, such as within the IMU 506. The bottleneck model 904 represents a first portion of a CNN. The bottleneck model 904 includes an input 904-1 (e.g., the sensor data input 803 and/or the run features 805), which gets convolved, via the convolution layer 904-2. The max pool layer 904-3 then applies a down sampling operation to the feature maps generated by convolutional layer 904-2. The output of the max pooling layer 904-3 then gets convolved again, via the convolutional layer 904-4, after which another round of max pooling and convolution occur again via the max pooling layer 404-4 and the convolutional layer 904-6. For example, the input at 904-1 may first be a shape of (256, 3), which gets convolved (via convolutional layer 904-2) into shape (256, 34), after which the max pooling layer 904-3 and the convolutional layer 904-4 reduce dimensionality to shape (42, 4). The max pooling layer 404-5 and the convolutional layer(s) 404-6 then perform another iteration to reduce the shape again, such as to (7, 4). The max pooling layer 904-7 then performs a flattening function (e.g., via the flatten layer 811) to flatten or convert 2D data into a 1D vector, represented at 904-8, which may now be at a substantially reduced shape, such as (4, 1).

The flattened output 904-8 thus represents a condensed estimation, such as a condensed representation of the estimated force curve 610, and/or a condensed representation of the estimate gait metrics 612 of FIG. 6. In response to the flattening layer producing the flattened output 904-8, some aspects terminate the bottleneck model 904 at the flattened output 904-8 (e.g., the bottleneck layer). For instance, the flattened output 904-8 may only have four values, whereas a full force curve may have 128 values. This saves storage space on the article of footwear storage 902. More storage space allows for more force curves or other metrics to be stored to the article of footwear storage 902 than would otherwise be possible. This also makes it more computationally more efficient to transfer the force curve or other metrics over the network(s) 910. For example, network latency is reduced (and potential savings in bandwidth) when data is transferred, over the network(s) (e.g., a LAN or WAN), to the user device 912 and/or another network device, such as a cloud service node because there is less data to transmit.

Terminating a model early means to stop the forward pass of the network at an intermediate layer (e.g., a bottleneck layer, such as a flattening layer), before reaching the final output layer (i.e., the dense layer 904-4). To terminate such model early, some aspects call the intermediate layer's index or name within the CNN. Then some aspects can access the layers of the CNN model using deep learning framework's API (e.g., in Python, commonly used libraries include TensorFlow and PyTorch). In some aspects, a new model, such as the dense model 906 is created. The bottleneck model 904 is a subset or second portion of the dense model 906. In some aspects, the dense model 906 includes all layers from the input layer up to and including where termination occurs (i.e., layer 904-1, 904-2, 904-3, 904-4, 904-5, 904-6, 904-7, and 904-8). However, the dense model 906 additionally includes the dense layer 904-9, which the bottleneck model 904 does not have. In some aspects, however, the dense model 906 alternatively only contains the flattened output 904-8 and the dense layer 904-9 (and none of the other layers in the bottleneck model 904), as illustrated in FIG. 9. A dense layer (e.g., a fully connected layer) of a CNN is a type of layer that is fully connected, meaning that each neuron in the dense layer is connected to every neuron in the previous layer. Each connection between neurons has a learned weight and bias associated with it. These weights and biases may be learned during the training process through backpropagation and gradient descent, allowing the network to adapt and model complex relationships in the data. In some aspects, a non-linear activation function, such as ReLU (Rectified Linear Unit) or Sigmoid, is applied to the output of each neuron in the dense layer 904-9. This introduces non-linearity into the network, allowing it to model more complex functions.

Accordingly, the final dense layer 904-9 is then run at the user device 912 (and/or in the cloud) to generate, for example, the full 128 sample force curve or other gait metrics. The 4 value bottleneck output 904-8 represents the full force curve (but in a lossless manner). In other words, if it is desired to store force curves or other metrics to the article of footwear storage 902, 128 floating values would typically have to be stored (e.g., per stride). However, with the bottleneck model 904, only 4 values, for example, would have to be stored, which represent the 128 values in a lossless manner. Accordingly, for example, if there is a request for the user device 912 to display a final predicted force curve or other metric, the bottleneck output 904-8 may be produced to a network interface at the article of footwear, which then transmits, over the network(s) 910, the bottleneck output 904-8. Responsively, the dense model 906 decodes the bottleneck output 904-8 to generate, via the dense layer 904-9, the decoded dense data (e.g., shape 128, 1), which is much more information than the output 904-8. In some aspects, if there is no such display or other transmission request, then there is no transmission of the output 904-8, over the network(s) 910, to the user device 912. Rather, all of the data is kept locally at the article of footwear storage 902. Accordingly, for example, there is a significant reduction in the storage required for strides or any time window (e.g., 4 floats per stride rather than 128).

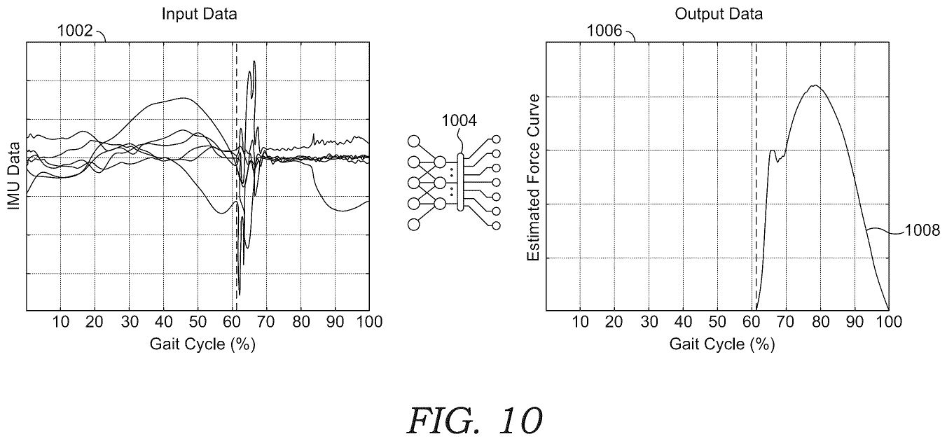

FIG. 10 is a schematic diagram illustrating, at a high level, a force curve estimation (or change in force estimation) based on processing IMU data, according to some aspects. FIG. 10 includes the input data 1002, the machine learning model 1004, and the output data 1006. In some aspects, the IMU data of the input data 1002 represents the sensor data as produced by the IMU 506 of FIGS. 5A and 5B. In some aspects, the machine learning model 1004 represents the neural network 705, the CNN 800, the bottleneck model 904, or the dense model 906. In some aspects, the output data 1006 represents the output 815 of FIG. 8, the force curve 406 of FIG. 4, the flattened output 904-8 of FIG. 9, the output of the dense layer 904-9, the estimated force curve 610 of FIG. 6, the inference prediction 709, and/or the training prediction 707 of FIG. 7.

As illustrated in FIG. 10 the input data 1002 is represented by a graph, where the Y-axis is represented by IMU sensor data, where each line represents a different axis (e.g., pitch, yaw, and roll) of one or more sensors. The X-axis refers to the gait cycle in percentage. In this way, it can be determined what the particular sensor data values are at a specific portion of a gait cycle or percentage. The gait cycle percentage refers to the division of a complete walking or running cycle into various phases or events, each expressed as a percentage of the entire cycle. This concept may be used in biomechanics and gait analysis to understand and quantify the different components of a person's stride. The gait cycle typically includes two main phases: stance phase and swing phase. These phases are further divided into specific events. The gait cycle is usually defined as starting when one foot makes initial contact with the ground (initial contact) and ending when the same foot contacts the ground again. The cycle can be divided into the following key events, and each event is expressed as a percentage of the entire gait cycle: Initial Contact (0%)—this is the moment when one foot first touches the ground to start a new stride. Loading Response is a phase that begins right after initial contact and includes the period when the body weight is transferred onto the stance limb. It typically spans from 0% to around 12% of the gait cycle. Midstance is a phase that follows the loading response and represents the period when the stance limb is fully supporting the body's weight. It typically ranges from about 12% to 31% of the gait cycle. Terminal Stance is a phase that marks the transition from midstance to the initiation of the swing phase. It usually occurs from around 31% to 50% of the gait cycle. Pre-Swing is a brief period that prepares the leg for the upcoming swing phase and spans from about 50% to 62% of the gait cycle. Initial Swing is the beginning of the swing phase, where the foot lifts off the ground and starts moving forward. It typically covers 62% to 75% of the gait cycle. Mid Swing phase is the middle of the swing phase and ranges from around 75% to 87% of the gait cycle. Terminal Swing occurs near the end of the swing phase, just before the foot prepares to make initial contact again. It usually spans from 87% to 100% of the gait cycle. The gait cycle percentage is useful for analyzing and understanding the timing and coordination of various events during walking and running. In some embodiments, 0% starts with Toe-Off, as opposed to “contact” as described above.

The input data 1002 is fed to the machine learning model 1004, which generates a score, inference, or prediction—i.e., the output data 1006—for the corresponding gait cycle percentage or portion (as represented in the X-axis) as illustrated in the input data 1002. The score, inference, or prediction is indicative of a corresponding estimated quantity of force or force change (as represented in the Y-axis) over the gait cycle, as represented by the force curve 1008. For example, FIG. 10 illustrates that for a given set of IMU sensor values at gait cycle percentage of around 80%, these sensor values are predicted to cause the highest quantity of force (i.e., the peak of the force curve 1008) relative to the other sensor values at other gait cycles.

### Example Methods

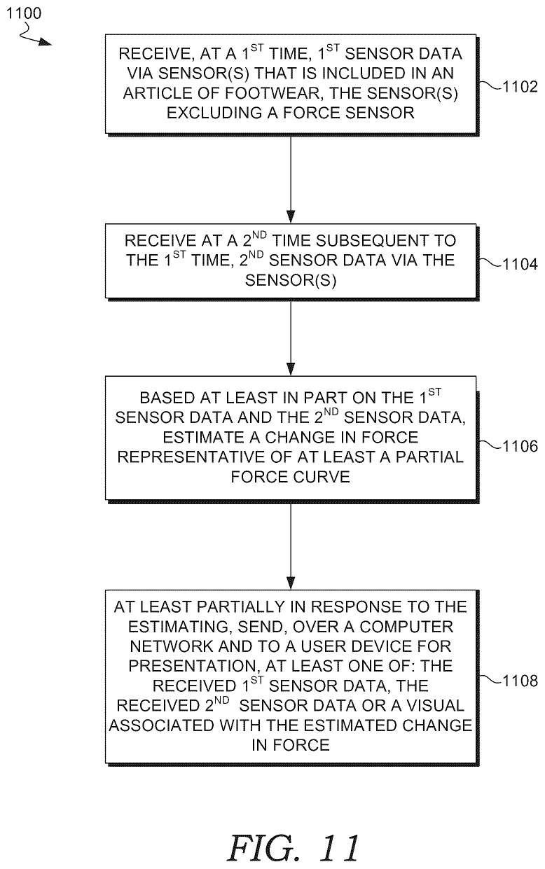

FIG. 11 is a flow diagram of an example process 1100 for estimating a change in force over at least one parameter, according to some aspects. The process 1100 (and/or any of the functionality described herein) may be performed by processing logic that comprises hardware (e.g., circuitry, hardware accelerator (e.g., AI accelerator), dedicated logic, programmable logic, microcode, etc.), software (e.g., instructions run on a processor to perform hardware simulation), firmware, or a combination thereof. Although particular blocks described in this disclosure are referenced in a particular order at a particular quantity, it is understood that any block may occur substantially parallel with or before or after any other block. Further, more (or fewer) blocks may exist than illustrated. Added blocks may include blocks that embody any functionality described herein (e.g., as described with respect to FIG. 1 through 19). The computer-implemented method, the system (that includes the wearable article and a processor), and/or a computer readable medium in association with a processor as described herein may perform or be caused to perform the process 1200 or any other functionality described herein. In some aspects, one or more processors of an article of footwear or system (e.g., that includes the article of footwear and/or a processor(s)) performs one or more of the processes 1100 through 1600 of FIGS. 11 through 16. Alternatively or additionally one or more processors stored to a server or backend device (e.g., system 1808 of FIG. 18) performs one or more of the processes 1100 through 16.

Per block 1102, some aspects receive, at a first time, first sensor data (e.g., the sensor data 602 of FIG. 6) via one or more sensors that is included in an article of footwear, where the one or more sensors exclude a force sensor. For example, an accelerometer in a shoe can measure velocity at a particular point in time corresponding to a swing phase of a gait cycle and then transmit such sensor value to an on-chip processor to be received by the processor. A “force sensor” (e.g., a force transducer or load cell) is a device used to directly measure the force applied to it. It converts mechanical force into an electrical signal that can be measured and interpreted directly as force (e.g., Newtons). For example, a force sensor can be a force plate, also known as a force platform, which is a specialized instrument used to measure the ground reaction forces (GRFs) exerted on it by an object or a person standing or moving on it. Other sensors, such as accelerometers and gyroscopes are not force sensors, as they do not directly measure force. Rather, for example, accelerometers measure linear acceleration, which is the rate of change of velocity (speed or direction) of an object along one or more axes. Gyroscopes measure angular velocity, which is the rate of rotation or change in orientation of an object about one or more axes. They provide information about the object's rotational motion and how it is changing direction. Is some aspects, the article of footwear (e.g., corresponding to heel strike) is in a first position at the first time.

In some aspects, the one or more sensors are included in any suitable wearable article (not just an article of footwear). For example, the one or more sensors can be included in an upper body garment (e.g., a shirt), a lower body garment (e.g., pants), a wrist band, helmet, armband, or any other suitable wearable article. In some aspects, the one or more sensors are included in an Inertial Measurement Unit (IMU), as described, for example by the IMU 506 of FIGS. 5A and 5B.

Per block 1104, some aspects receive, at a second time subsequent to the first time, second sensor data via the one or more sensors. For example, the accelerometer in the shoe can measure another velocity at a subsequent point in time corresponding to a heel-strike phase of the gait cycle and then transmit such sensor value to the on-chip processor to be received by the processor. In some embodiments, the article of footwear is in a second position at a second time subsequent to the first time (e.g., toe off). Per block 1106, based at least in part on the first sensor data and the second sensor data, some aspects estimate a change in force representative of at least a partial force curve (e.g. predicted force from time 1 to time 2 over a period that spans to time 5). The partial force curve may be estimated over at least one parameter, such as one or more of, time, a gait cycle, a gait percentage (e.g., as illustrated in FIG. 10), or other activity type. In some aspects, estimating a change in force inherently includes estimating, predicting, or computing force at different points in time or at a certain event. In some aspects, block 1104 (and the process 1100) alternatively represents estimating force instead of a change in force.

In some aspects, the estimating at block 1106 is based on using a Convolutional Neural Network (CNN), as described, for example, in FIG. 8 and/or FIG. 9. In some aspects, a first portion of the CNN is stored to a storage device (e.g., the article of footwear storage 902) at the article of footwear, where the first portion includes an intermediate layer (e.g., a bottleneck layer or flattening layer) that produces an output (e.g., the flattened output 904-8) indicative of the estimate. An “intermediate” layer is any layer within the model that is not the last layer and not the first layer. For example, in the context of neural networks, the intermediate layer can be a hidden layer, but not the input layer or the output layer. In various aspects, the first portion does not include a dense output layer (e.g., the dense layer 904-9 of FIG. 9), but a second portion of the CNN is stored to a user device (e.g., the user device 912) and does include the dense output layer (e.g., as illustrated in the dense model 906).

Some aspects add one or more run features (e.g., the run features 805 or 813 of FIG. 8) to one of, a first input into a convolutional layer (e.g., the convolutional layer 807) at the CNN or a second input to a flattening layer (e.g., the flatten layer 811 of FIG. 8) at the CNN. In this way, the estimating is based at least in part on the adding of the one or more run features. Examples of this are described with respect to the force curve estimation component 608 estimating a force curve 610 based on ingesting the estimated gait metrics 612 produced by the gait metric estimation 606.

In some aspects, the estimation at block 1106 is based only selected axes or sensor data processed (e.g., by a machine learning model) from an accelerometer and gyroscope (e.g., a 3-axis input). For example, an IMU can include an accelerometer that provides a first portion of the sensor data along a longitudinal axis and a vertical axis. However, the estimation may not be based on (e.g., not fed to the model) the accelerometer providing the first portion of sensor data along a mediolateral axis. Longitudinal or Anteroposterior Axis (X-axis) measures acceleration in the front-to-back direction, parallel to the sagittal plane of the body. Positive acceleration along this axis would correspond to forward motion, while negative acceleration would correspond to backward motion. Transverse or Mediolateral Axis (Y-axis) measures acceleration in the side-to-side direction, parallel to the frontal plane of the body. Positive acceleration along this axis would correspond to motion to the right, while negative acceleration would correspond to motion to the left. Vertical or Craniocaudal Axis (Z-axis) measures acceleration in the up-and-down direction, perpendicular to the ground. Positive acceleration along this axis represents upward motion, and negative acceleration represents downward motion.

Continuing with this example, the gyroscope may provide a second portion of the sensor data via a pitch axis, but the estimation at block 1106 is not based on the gyroscope providing the second portion via a yaw axis or a roll axis. Yaw refers to the rotation of an object around its vertical axis, which is typically aligned with the direction of gravity. This movement is akin to turning left or right, causing a change in the object's (e.g., IMU) heading or direction without tilting up or down. Pitch refers to the rotation of an object (e.g., IMU) around its lateral or transverse axis. This movement involves tilting the object up or down, causing it to climb or descend. Roll refers to the rotation of an object around its longitudinal or roll axis. This movement involves tilting the object from side to side, similar to the motion of rolling a ship or an aircraft about its long axis.

In some aspects, the estimating at block 1106 is based on processing only a fixed window (e.g., a particular data chunk and/or time sequence) of sensor data indicative of a gait phase of a full gait cycle, where other sensor data indicative of other portion of the full gait cycle are not processed for the estimation. For example, a data structure may map a particular accelerometer range or threshold with a particular gait phase, such as touchdown. “Touchdown” refers to the moment when the foot or article of footwear of the leading leg makes initial contact with the ground. It is one of the core events that occur during the stance phase of the gait cycle, which is the period when the foot is in contact with the ground. In an illustrative example, in response to the accelerometer data spiking or reading over a threshold, various aspects transfer a predefined window (e.g., 1 second before and 1 second after) of the accelerometer data in relation to the spike and feed that data into a model (e.g., the CNN 800 for further processing). In some aspects, for example, such window of sensor data is the only data included in the sensor data 602. With respect to the “spike” described herein, a typical accelerometer sensor might, for example, measure accelerations ranging from −2 g to +2 g, where 1 g represents the standard gravitational acceleration on Earth (approximately 9.81 meters per second squared). This means that the sensor value at a spike can exceed 2 g or even reach significantly higher values, depending on the intensity of the event. For example, at touchdown, the accelerometer may record a spike that briefly exceeds a 2 g threshold (e.g., between 5 g and 10 g) based on experiencing a sharp deceleration when the article of footwear hits the ground. This may responsively trigger aspects to capture a threshold quantity or time sequence of sensor data values measured before and/or after the spike or threshold for processing. This is intuitive because touchdown and other gait phases are highly indicative of estimated ground force, whereas other gait phases may not be as indicative of such force.

Based at least in part on one of the first sensor data or the second sensor data, some aspects classify an activity type that the wearer of the article of footwear has engaged in. In this way, the estimation at block 1106 is based at least in part on the classification of the activity type. Examples of this are described with respect to the gait segmentation component 604, which is generally responsible for detecting, estimating, classifying, predicting, or otherwise computing which activity type a wearer is in. In some aspects, the activity type includes at least one of, running, walking, jogging, jumping, landing, or a gait phase, of an entire gait cycle. In some aspects, the classification of the activity type is based on using a piecewise model as described, for example, with respect to the gait segmentation component 604 and the force curve estimation component 608, where, for example, a “running” model can be used to estimate force.

Per block 1108, at least partially in response to the estimating, some aspects send, over a computer network (e.g., the network(s) 910) and to a user device for presentation, at least one of: the received first sensor data, the received second sensor data, or a visual associated with the estimated change in force over the at least one parameter. In some aspects, the visual includes a ground force curve, such as illustrated by the curve 1008, the estimated force curve 610, or the force curve 406, or the output data 1006. In some aspects, the visual includes an immersive technology element. An immersive technology element, as described herein, refers to an element (e.g., a virtual data element) of immersive technology. Immersive technology refers to technologies that create a sense of immersion or presence for users, typically by engaging multiple senses to make them feel as though they are part of a computer-generated or augmented environment. Immersive technologies can include virtual reality (VR), augmented reality (AR), and/or mixed reality (MR). For example, referring back to FIG. 1, in some aspects, the immersive technology element refers to any of the screenshot 100 of FIG. 1, such as the visual data 104 or 106, which is indicative of growing Godzilla legs in response to detecting that the force, change in force, or other gait metric (e.g., foot strike angle) exceeds a threshold.

In some aspects, the visual additionally or alternatively represents a gaming element of a video game. For example, referring back to FIG. 2, in some aspects, the gaming element includes any of the screenshot 200, such as the visual data 202 corresponding to the T-rex chasing the avatar 201 in response to a predicted force (or change in force) exceeding a threshold. Other gaming embodiments may alternatively be employed. For example, the gaming element could include a gamer (or visual data representing its avatar) shooting a rocket when they stump their feet (or article of footwear 500 of FIG. 5A) over a force (or other metric) threshold, where the rocket height is directly proportional to the quantity of predicted force exhibited. In another example, the gaming element can be included in a basketball game. If an avatar that represents an athlete dunks a ball with a threshold quantity of force, change in force, or leaping strength (and/or other gait metric exceeds a threshold), upon landing on the ground, a broadcast system may overlay the video feed with a graphic of an explosion emanating from the athlete on the court surface itself, as described, for example, in US patent application number US20220279891 A1, which is incorporated by reference in its entirety.

In some aspects, the visual additionally or alternatively includes a user interface element (e.g., any element within the screenshot 400). For example, the user interface element can include an element that warns a user when the force exceeds a threshold. An example of this is the popup window 408 of FIG. 4. Some aspects determine that at least one of, the first sensor data, the second sensor data, or an estimated force exceeds a threshold. Based on such determination, some aspects send a notification to a user device. For example, referring to the popup window 408, some aspects cause display of the popup window 408 when the estimated VGRF exceeds a ground force threshold.

Based at least in part on the estimating of the change in force over at least one parameter (or other metric), some aspects cause the article of footwear to apply a resist or assist force as described, for example, with respect to FIG. 3B. Based at least in part on the estimating of the change in force over at least one parameter (or estimating some other metric), some aspects classify a lower limb gesture, such as a stomp, a kick, or a jump, as described in more detail below.

Based at least on the estimated change in force (or other estimated metric), some aspects (e.g., the CNN 800) estimate a shape of a force curve. Some aspects additionally scale this shape output using a separate regression model (e.g., separate from the CNN 800). A regression model scales the shape of a curve by fitting a mathematical function or equation to a set of data points. The specific shape of the curve that the model scales depends on the type of regression model used. There are various types of regression models, and each has its own way of scaling the shape of the curve. To estimate the shape of the curve, regression models may minimize a cost function (e.g., mean squared error) by adjusting their parameters (e.g., coefficients in linear regression or weights in neural networks) to best fit the observed data points. The choice of the specific regression model and its parameters will affect how well it can capture the shape of the underlying curve in the sensor data.

In an example illustration, linear regression models estimate a straight line (a linear relationship) between the independent variable(s) and the dependent variable. The equation of a simple linear regression model is y=mx+b, where “y” is the dependent variable, “x” is the independent variable, “m” is the slope of the line, and “b” is the y-intercept. The shape of the curve is a straight line. Polynomial regression models estimate curves of higher degrees (e.g., quadratic, cubic, etc.). These models can capture more complex relationships between variables by fitting a polynomial equation to the data. The equation might look like y=a+bx+cx² for a quadratic curve. The shape of the curve can be a parabola or a more complex polynomial shape, depending on the degree of the polynomial. Nonlinear regression models estimate curves that do not follow a simple linear or polynomial pattern. These models use various types of functions, such as exponential, logarithmic, sigmoid, or other custom functions, to capture the shape of the curve. The specific function chosen depends on the nature of the data and the problem. Other models may additionally or alternatively be used to approximate the shape of a curve, such as spline regression models, decision trees and random forests, neural networks

In some aspects, estimating or scaling the shape of the force curve includes one or more smoothing operations. Smoothing is a technique used to reduce noise and emphasize patterns or trends in the input data, often by estimating a smoother curve. Smoothing techniques are applied to help reveal the underlying structure or relationships in the data and can be considered a form of regression in some cases. Smoothing methods, like moving averages, kernel smoothing, or loess (locally weighted scatterplot smoothing), aim to create a smoother representation of data points, such as different force curve estimations. These techniques involve calculating weighted averages of nearby data points, which effectively results in a smooth curve that captures trends and removes noise. This is similar to a regression model's goal of estimating a curve that represents the relationship between variables. Some regression models can also be viewed as smoothing techniques. For example, kernel regression (a type of non-parametric regression) uses a kernel function to estimate a smoother curve through the data. This curve is not constrained to be linear or follow a specific polynomial form, making it a flexible smoothing approach.

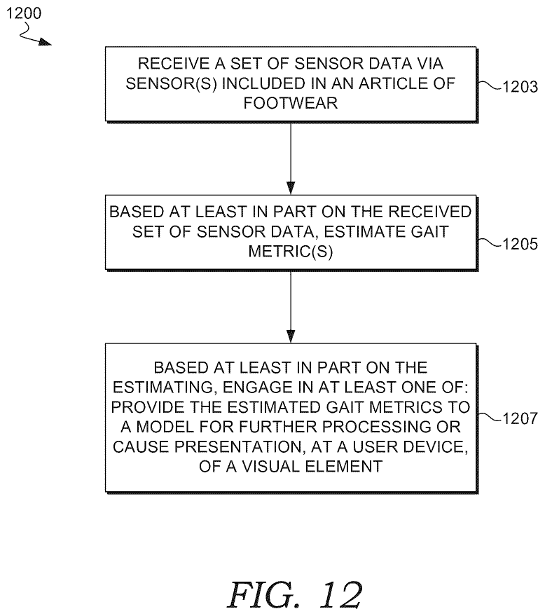

FIG. 12 is a flow diagram of an example process 1200 for estimating one or more gait metrics, according to some aspects. Per block 1203, some aspects receive a set of sensor data via sensor(s) included in an article of footwear. In some aspects, block 1203 includes all the functionality described with respect to block 1102 of FIG. 11. Per block 1205, based at least in part on the received set of sensor data, some aspects estimate one or more gait metrics. Examples of this are described with respect to the gait metric estimation component 606 estimating the estimated gait metrics 612 based on analyzing the sensor data 604. For example, estimated gait metrics can be strides (quantity of), activity type (run), speed (e.g., mph), Gait Cycle Time (in ms), stride length (in m), cadence (steps per minute), foot strike angle (in degrees), VGRF (Vertical Ground Reaction Force) average, VGRF peak, VGRF impulse, and/or VGRF instantaneous LR.

Quantity of strides (e.g., number of steps while running or walking), for example, can be predicted based on a pattern of accelerometer data spiking at a threshold over a time interval (e.g., 4 spikes are indicative of 4 strides). In some aspects, activity type is estimated via the gait segmentation component 604 as described in FIG. 6. Speed is the rate at which an object (e.g., an IMU) is changing position with respect to time. Gait Cycle Time refers to the time it takes for one complete cycle of human walking or running, starting from one foot's initial contact with the ground (heel strike) through a full step until the same foot makes contact with the ground again. The gait cycle is typically divided into two phases: the stance phase (when the foot is in contact with the ground) and the swing phase (when the foot is off the ground). Gait cycle time is the time taken to complete both the stance and swing phases for one leg. For instance, an IMU can monitor the acceleration patterns, via an accelerometer, and a processor can identify key events in the gait cycle, such as heel strikes and toe-offs to determine the gait cycle time and other temporal parameters.

Stride length refers to the distance covered in a single stride, which is the linear distance between two consecutive foot contacts of the same foot during walking or running. A processor, for example, can analyze the acceleration signals to identify key events in the gait cycle, such as the heel strike and toe-off. The processor can then calculate the time between these events to determine the duration of a stride and then multiply the duration of a stride by the average velocity to estimate stride length. The average velocity can be calculated based on the linear distance traveled over the duration of the stride. Cadence or steps per minute can be measured by counting how many time accelerometer or gyroscope data spike over a threshold in one minute.

Foot strike angle, also known as the foot strike pattern, refers to the angle (e.g., in degrees) at which the foot contacts the ground during walking, running, or other forms of locomotion. It describes the orientation of the foot relative to the ground when it first makes contact during the gait cycle. A gyroscope, for example, can measure the angular changes as the article of footwear moves through the gait cycle. A processor can then analyze the gyroscope data to identify the angle at which the foot makes initial contact with the ground. This angle represents the foot strike pattern. Different foot strike patterns, such as heel strike, midfoot strike, or forefoot strike, will result in different angular orientations at initial contact. Some embodiments can categorize the foot strike pattern based on the angle measured by the gyroscope. For example, a large negative angle might indicate a heel strike, while a smaller or positive angle might indicate a midfoot or forefoot strike. It is understood, however, that this example of negative and positive angles are representative only. For example, in some embodiments a negative angle is indicative of a forefoot strike and a positive angle is indicative of a heel strike.

Per block 1207, based at least in part on the estimating, some aspects engage in at least one of, provide the estimated gait metrics to a model for further processing or cause presentation, at a user device, of a visual element. For example, the providing of the estimated gait metrics is described in FIG. 6, where the estimated gait metrics 612 are returned to the force curve estimation component 608. In some aspects, such visual element includes any one of the visual elements within the screenshot 400, such as the gait metrics 402.

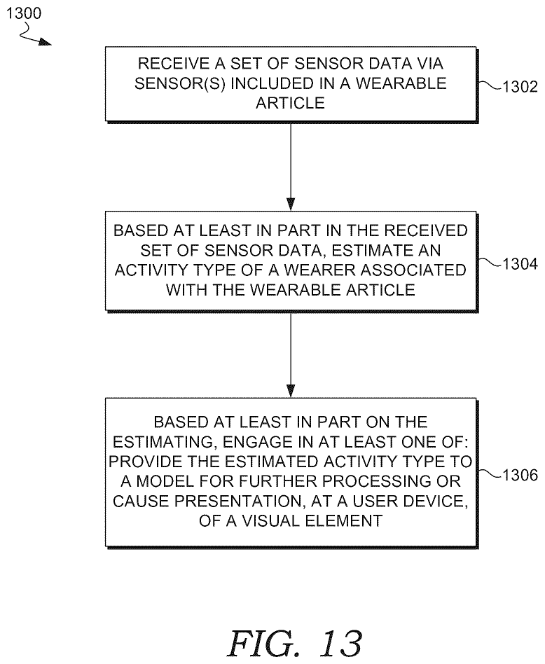

FIG. 13 is a flow diagram of an example process 1300 for estimating an activity type of a wearer associated with a wearable article, according to some aspects. Per block 1302, some aspects receive a set of sensor data via sensor(s) included in a wearable article (e.g., an upper body garment, a wrist ban, or an article of footwear). In some aspects, block 11302 includes all the functionality described with respect to block 1102 of FIG. 11.

Per block 1304, based at least in part on the received set of sensor data, some aspects estimate an activity type of a wearer associated with the wearable article. In some aspects, activity type is estimated via the gait segmentation component 604 that computes or classifies which activity type a wearer is in as described in FIG. 6. Per block 1306, based at least in part on the estimating, some aspects engage in at least one of, provide the estimated activity type to a model for further processing or cause presentation, at a user device, of a visual element. For example, the providing of the estimated activity type is described in FIG. 6, where the estimated activity type(s) is transmitted from the gait segmentation component 604 to the force curve estimation component 608. In some aspects, such visual element includes any one of the visual elements within the screenshot 400, such as the “activity . . . run” indicia located in the gait metrics 402.

FIG. 14 is a flow diagram of an example process 1400 for classifying a gesture of a wearer, according to some aspects. Per block 1402, some aspects receive a set of sensor data (e.g., the sensor data 602 of FIG. 6) via sensor(s) included in a wearable article (e.g., an upper body garment, a wrist ban, or an article of footwear). In some aspects, block 1402 includes all the functionality described with respect to block 1102 of FIG. 11.

Per block 1404, based at least in part on the received set of sensor data, some aspects classify a gesture of a wearer of the wearable article. For example, accelerometer and gyroscope sensor data can be used to predict a lower limb gesture, such as a stomp, a kick, or a jump. Machine learning algorithms, such as decision trees, support vector machines, or deep learning models like convolutional neural networks (CNNs) and recurrent neural networks (RNNs), for example, can be trained on labeled datasets of sensor data, where the corresponding gestures are the labels. These algorithms learn to recognize patterns in the data that correspond to specific gestures. During the training phase, for example, the algorithm is exposed to a dataset of labeled examples, where each example includes sensor data and the associated gesture label. The algorithm learns to associate patterns in the data with the corresponding gestures. In some aspects, such classified gesture is performed by the gait segmentation component 604, as described in FIG. 6, where the activity type is, for example, a classified gait gesture.

Per block 1406, at least partially responsive to the classifying, some aspects trigger a particular action. Once the algorithm identifies the gesture, for example, it can trigger a specific action or response in the device or application. For example, recognizing a particular gesture could control a character in a video game, navigate a user interface, or otherwise perform a specific function in a computer application. Referring back to FIG. 1, for example, in response to detecting classifying the avatar 102's leg motion as a “stomp,” some aspects warp pixels of the visual data 108 representative of a car, which indicates the car 108 is smashed. The particular action may alternatively or additionally include sending, or a computer network and to a user device for presentation, at least one of, the set of sensor data or an indication of the classified gesture.

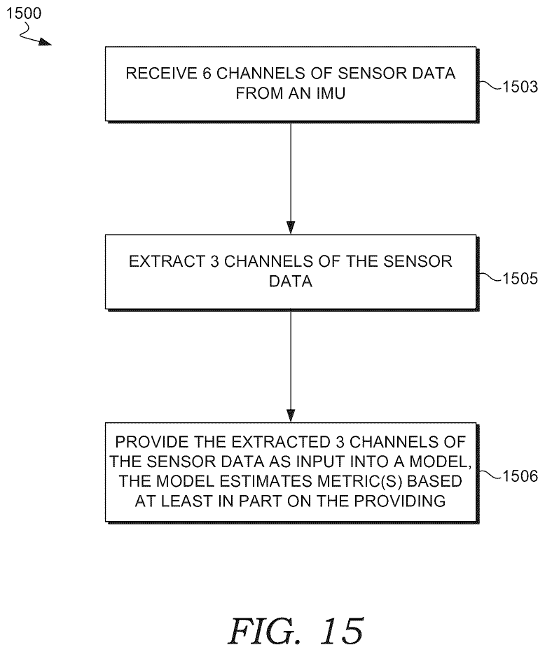

FIG. 15 is a flow diagram of an example process 1500 for extracting and providing 3 channels of sensor data as input to a model for estimation, according to some aspects. Per block 1503, some aspects first receive (e.g., store in computer memory) 6 channels (also referred to herein as “6-axis”) of sensor data from an IMU. For example, each axis or channel may correspond to 3 channels of an accelerometer (longitudinal, vertical, and mediolateral) and 3 channels of a gyroscope (pitch, yaw, and roll).

Per block 1505, some aspects extract 3 channels of the senor data. For example, extraction can include accessing or retrieving, from the computer memory, 2 channels from the 3-channel accelerometer sensor data—the longitudinal axis and vertical axis sensor data (while discarding the mediolateral sensor data)—and 1 channel from the gyroscope data-only the pitch axis sensor data (but not accessing the yaw or roll axis sensor data).

Per block 1506, some aspects provide the extracted 3 channels of the sensor data as input into a model (e.g., the neural network 705 of FIG. 7, the CNN 800 of FIG. 8, or the bottleneck model 904 of FIG. 9), where the model estimates one or more metrics (e.g., force, changes in force, any of the gait metrics described herein, activity type classifications, etc.) based at least in part on the providing. For example, referring back to FIG. 7, the neural network 705 may make an inference 709 of a force curve based on analyzing the extracted 3 channels of sensor data included in the sensor data A of the deployment input(s) 703 (and/or analyzing the activity type classification B or the gait metrics of the deployment input(s) 703).

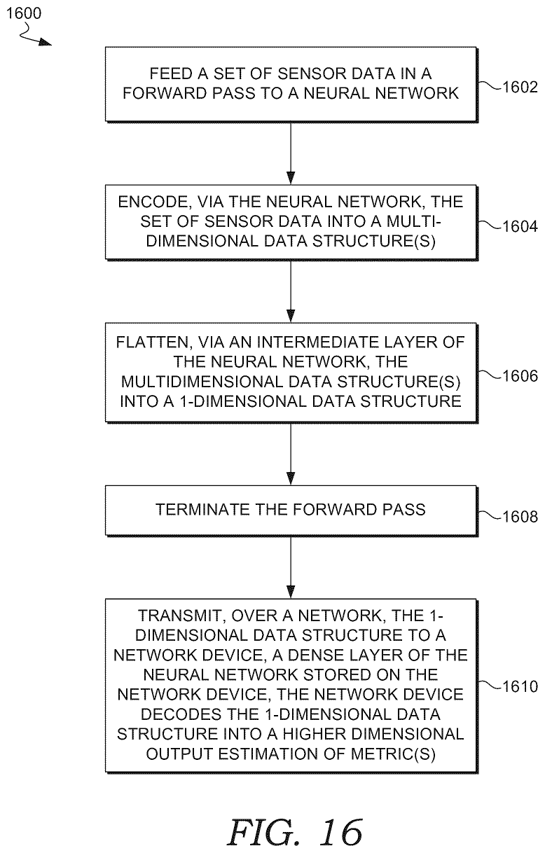

FIG. 16 is a flow diagram of an example process 1600 for making an estimation of one or more metrics based on encoding and decoding model data via different devices, according to some aspects. In some aspects, the process 1600 represents the functionality described with respect to FIG. 9. Per block 1602, some aspects feed a set of sensor data (e.g., the sensor data 602 of FIG. 6) in a forward pass to a neural network (e.g., the bottleneck model 904). Per block 1604, some aspects then encode, via the neural network, the set of sensor data into one or more multi-dimensional data structures. Examples of this are described, for example, with respect to the convolutional layer 807 of FIG. 8, which produces a feature map, such as a 2D or 3D tensor.

Per block 1606, some aspects flatten, via an intermediate layer of the neural network, the multidimensional data structure into a 1-dimensional data structure, such as a vector. Examples of this are described with respect to the flatten layer 811, and the max pooling layer 904-7 to produce the flattened output 904-8.

Per block 1608, some aspects terminate the forward pass, as described, for example with respect to FIG. 9 and perform an early termination at the flattened output 904-8. Per block 1610, some aspects transmit, over a network (e.g., the network(s) 910), the 1-dimensional data structure (and/or other command, such as a command to produce an estimated output) to a network device (e.g., the user device 912, a server, or a cloud service node), where a dense layer of the neural network stored on the network device decodes the 1-dimensional data structure into a higher dimensional output estimation of one or more metrics (e.g., estimated force, change in force, estimated gait metrics, estimated activity type). Examples of this are described in FIG. 9 where the dense layer 904-9 outputs a higher dimensional output estimation (e.g., 128 floats), as opposed to the 1-dimensional data structure (e.g., which may only include 4 floats).

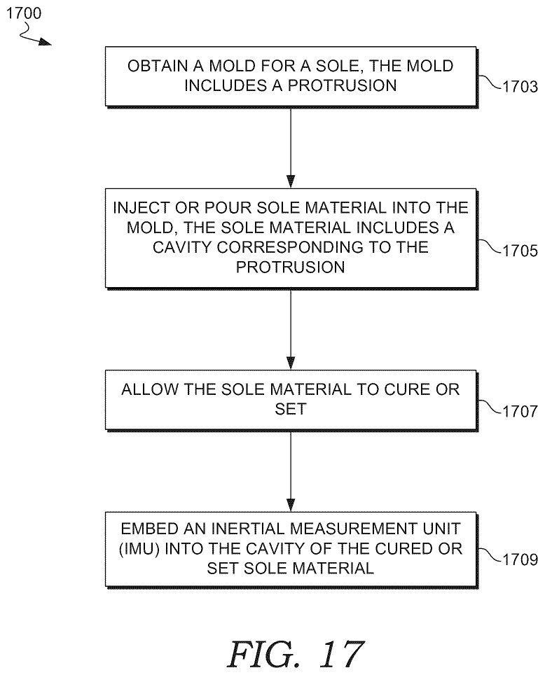

FIG. 17 is an example process 1700 for manufacturing an article of footwear with an embedded IMU, according to some aspects. In some aspects, the process 1700 is used to manufacture the article of footwear 500 of FIG. 5A and FIG. 5B. Per block 1703, some aspects obtain a mold for a sole (e.g., a midsole), where the mold includes a protrusion. For example, the protrusion may be a volume of space that is a negative for a cavity. Example sizes of the protrusion include 38.0 mm×24.0 mm×9.7 mm (greater or less than 5%) or (18 mm×16 mm×4 mm (greater or less than 5%).

Per block 1705, some aspects inject or pour sole material into the mold, where the sole material includes a cavity corresponding to (or is the result of) the protrusion. For instance, since the protrusion is a volume of space, the sole material does not enter into the protrusion or volume of space within the protrusion and therefore forms outside of such protrusion, thereby creating a cavity.

Per block 1707, some aspects allow the sole material to cure or set. The curing or setting time for the midsole material of an article of footwear can vary significantly depending on the type of material used and the specific manufacturing process. Different materials, such as EVA foam, polyurethane, or rubber, may have different curing times. Additionally, the curing process can be influenced by factors like temperature, humidity, and the additives or chemicals used in the material. For examples, the curing time for EVA foam midsoles may range from 12 to 24 hours. However, the exact curing time can be adjusted by manufacturers based on their requirements. In another example, the curing time for Polyurethane (PU) midsoles may be within a range of 4 to 12 hours.

Per block 1709, some aspects then embed or position an Inertial Measurement Unit (IMU) into the cavity of the cured or set sole material. In various aspects, the IMUs are substantially the same size as the cavity so as to provide a tight friction fit (so as to allow little to no movement). For example, the IMUs can be a size of 38.0 mm×24.0 mm×9.7 mm (greater or less than 5%) or 18 mm×16 mm×4 mm (greater or less than 5%). In some aspects, additional manufacturing steps occur. For example, as described with respect to FIG. 5A, some aspects seal the midsole 504 (or the cavity) to protect the IMU 506 from moisture, impact, and other potential sources of damage. For example, sealing can include filling in the cavity 504-1 with additional EVA foam such that the IMU 506 is completely encompassed or surrounded by the midsole 502 (e.g., there is no opening for the IMU 506 to fall out of). This can be done using a sealant or cover that is also integrated into the midsole 504-1. Once the midsole 504 is ready, it can be incorporated into the rest of the shoe during the shoe manufacturing process, following the typical steps for stitching and assembly.

### Example Computing Environment

FIG. 18 is a block diagram illustrating an example computing environment 1800 for estimating one or more metrics from one or more sensors in an article of footwear 1805, according to some aspects. It should be understood that this and other arrangements described herein are set forth only as examples. Other arrangements and elements (e.g., machines, interfaces, functions, orders, and groupings of functions, etc.) can be used in addition to or instead of those shown, and some elements can be omitted altogether. Further, many of the elements described herein are functional entities that can be implemented as discrete or distributed components or in conjunction with other components, and in any suitable combination and location. Various functions described herein as being performed by one or more entities can be carried out by hardware, firmware, and/or software. For instance, various functions can be carried out by a processor executing instructions stored in memory.

The computing environment 1800 is an example of a suitable architecture or operating environment for implementing certain aspects of the present disclosure. Among other components not shown, the computing environment 1800 includes a user device 1802, an article of footwear 1804, and a wearable article processing system 1808. Each of the user device 1802, wearable article 1804, and the wearable article processing system 1808 can comprise one or more computer devices, such as the computing device 200 of FIG. 20, discussed below. As illustrated in FIG. 18, the user device 1802, the wearable article 1804, and wearable article processing system 1808 can communicate via a network 1806 (e.g., the network(s) 910 of FIG. 9), which can include, without limitation, one or more local area networks (LANs) and/or wide area networks (WANs). Such networking environments are commonplace in offices, enterprise-wide computer networks, intranets, and the Internet. It should be understood that any number of client devices and server devices can be employed within the computing environment 1800 within the scope of the present technology. Each can comprise a single device or multiple devices cooperating in a distributed environment. For instance, the wearable article processing system 1808 can be provided by multiple server devices or cloud nodes collectively providing the functionality of the wearable article processing system 1808 as described herein. Additionally, other components not shown can also be included within the network environment. In some aspects, the wearable article processing system 1808 is included in the user device 1802.

The user device 1802 (e.g., the user device 912) and/or the article of footwear 1804 can be a client device on the client side of computing environment 1800, while the wearable article processing system 1808 can be on the server side of computing environment 1800. The wearable article processing system 1808 can comprise server-side software designed to work in conjunction with client-side software on the user device 1802 and/or the article of footwear 1804 so as to implement any combination of the features and functionalities discussed in the present disclosure. For instance, the user device 1802 can include an application for interacting with the wearable article processing system 1808. The application can be, for instance, a web browser or a dedicated application for providing functions (e.g., the screenshot 400 of FIG. 4), such as those described with respect to FIGS. 11-16. This division of computing environment 1800 is provided to illustrate one example of a suitable environment, and there is no requirement for each implementation that any combination of the user device 1802 and the wearable article processing system 1808 remain as separate entities. While the computing environment 1800 illustrates a configuration in a networked environment with a separate user device and wearable article processing system, it should be understood that other configurations can be employed in which components are combined. For instance, in some configurations, a user device can also provide capabilities of the technology described herein.

The user device 1802 can comprise any type of computing device capable of use by a user. For example, in one aspect, the user device can be the type of computing device 8 described in relation to FIG. 20 herein. By way of example and not limitation, the user device 1802 can be embodied as a personal computer (PC), a laptop computer, a mobile or mobile device, a smartphone, a tablet computer, a smart watch, a wearable computer, handheld communications device, gaming device or system, entertainment system, vehicle computer system, embedded system controller, appliance, consumer electronic device, workstation, or any combination of these delineated devices, or any other suitable device. A user can be associated with the user device 1802 and can interact with the wearable article processing system 1808 via the user device 1802.

In some aspects, the wearable article processing system 1808 is configured to receive at least a portion of sensor data (e.g., the sensor data 5602) from the article of footwear 1808 (or more precisely the IMU1804-5) in order to interpret the sensor data and provide corresponding estimations to the user device 1802 (e.g., the estimated gait metrics 612 and/or the estimated force curve 610 of FIG. 6). Alternatively or additionally, in some aspects, the IMU 1804-8 of the article of footwear 1804 performs at least some of these estimations, as described for example, with respect to the article of footwear storage 902 of FIG. 9. Alternatively or additionally, in some aspects, the user device 1802 performs at least a portion of these estimations, as described, for example, with respect to the dense model 906 of the user device 912 of FIG. 9. Likewise, in some aspects, the article of footwear 1804 performs the processes 1100, 1200, 1300, 1400, 1500, and/or 1600. Alternatively or additionally, the wearable article processing system 1800 performs one or more of these processes. Alternatively or additionally, the user devices 1802 performs some each or of these processes.

The wearable article processing system 1808 can be implemented using one or more server devices, one or more platforms with corresponding application programming interfaces, cloud infrastructure, and the like. In one aspect, the functions performed by components of the wearable article processing system 1808 are associated with one or more applications, services, or routines. In particular, such applications, services, or routines can operate on one or more user devices or servers, be distributed across one or more user devices and servers, or be implemented in the cloud. Moreover, in some aspects, these components of the wearable article processing system 1808 can be distributed across a network, including one or more servers and client devices, in the cloud, and/or can reside on a user device. Moreover, these components, functions performed by these components, or services carried out by these components can be implemented at appropriate abstraction layer(s) such as the operating system layer, application layer, hardware layer, etc., of the computing system(s). Alternatively, or in addition, the functionality of these components and/or the aspects of the technology described herein can be performed, at least in part, by one or more hardware logic components. For example, and without limitation, illustrative types of hardware logic components that can be used include field-programmable gate arrays (FPGAs), application-specific integrated circuits (ASICs), application-specific standard products (ASSPs), system-on-a-chip systems (SOCs), complex programmable logic devices (CPLDs), etc. Additionally, although functionality is described herein with regards to specific components shown in example computing environment 1800, it is contemplated that in some aspects, functionality of these components can be shared or distributed across other components.

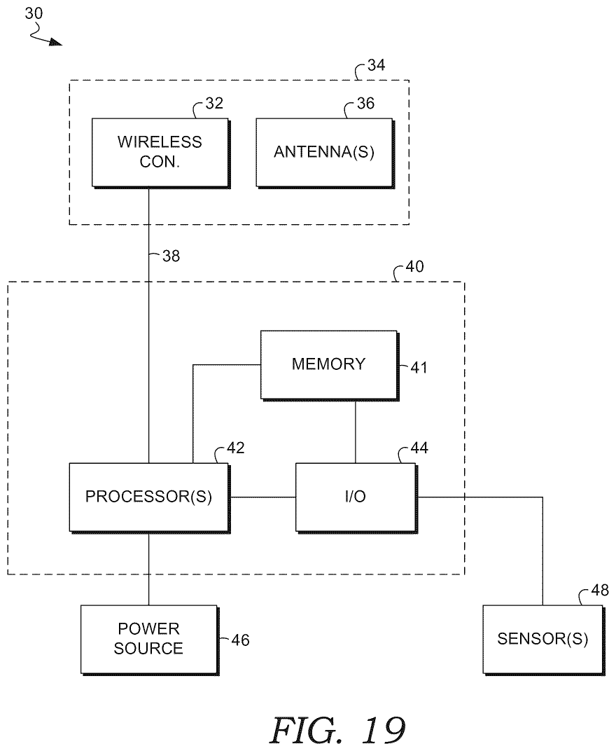

FIG. 19 is a block diagram 30 of the electronics of any wearable article described herein (e.g., the article of footwear 500 of FIG. 5A), according to some aspects. In some aspects, the block diagram 300 represents electronics of an IMU, such as the IMU 506 of FIGS. 5A and 5B. Although FIG. 19 illustrates various components that are included within a wearable article, in some aspects, some or each of the components are not part of the wearable article. For example, in some aspects, the electronics of a wearable article only includes the one or more sensors 48, the memory 41, and/or the wireless communication block 34 while the other components (e.g., the processor(s) 42) are hosted at a remote device, such as the wearable article processing system 2808 of FIG. 18. The block diagram 30 includes a wireless communication block 34, a control block 40, a power source 46, and one or more sensors 48 (e.g., an accelerometer and/or a gyroscope).

The power source block 46 includes a source of DC power, such as a battery, a super capacitor, and so forth, sufficient to provide power to the various other electronics. A battery may be a rechargeable battery or may be replaceable. Additionally or alternatively, the power source block 46 may include any of a variety of further power sources, including a piezoelectric generator or other source of power that may generate power though the movement or conventional use of the wearable article. The power source block 46 optionally includes additional componentry to boost or otherwise shift the power output of the source of DC power, such as a boost converter in an example, the power source block 46 includes a lithium ion battery configured to deliver between 3.0 and 4.2 Volts and a live (5) Volt boost converter.

The control block 40 receives power from the power source block 46 and controls the operation of the wireless communication block 34 and signals transmitted and received from the one or more sensors 48. The control block 40 include one or more processor(s) 42 (e.g., a microcontroller, a CPU, a GPU, and/or a TPU), an electronic memory 41 (e.g., the article of footwear storage 902), such as random-access memory (RAM), flash memory, data stream buffer(s), cache, and/or any suitable electronic memory known in the art, and an input/output block 44, among a variety of other components that may be desired or utilized. In an example, the control block 40 is a single system or system on a chip (e.g., a PCB substrate with circuitry components). In an example, the control block 40 includes an ATmega32U4 microcontroller by Atmel Corporation as the processor(s) 42 and related circuitry and/or by Lenoardo microcontroller board by Arduino Corporation, or any suitable controller or controller system. In some aspects, the processor(s) 42 performs the processes 1100 through 1600 of FIG. 11 through FIG. 16 described herein.

The sensor(s) 48 measures or detects sensor data, such as accelerometer or gyroscope readings and then forwards such data to the input/output block 44. When the sensor(s) 48 outputs a signal to the input/output block 44 of the control block 40, the input/output block 44 formats the signal(s) received from the sensor(s) and forwards the signal to the memory 41 (e.g., a stream buffer) and/or the processor(s) 42. The processor(s) 42 assesses the signal from the memory 41 or the input/output block 44 for various properties as desired, including, but not limited to, a time at which the output signal was sensed and a duration of the output signal, and/or interprets the signals via any of the estimations described herein, such as the estimated gait metrics 612, the estimated force curve 610, and/or estimated activity type. The processor(s) 42 may store such properties or estimations in the electronic memory 41 and/or act on the properties as appropriate.

The wireless communication block 34 (e.g., a network interface) includes one or more wireless antennas 36 and a wireless controller 32. The wireless antenna(s) 36 may each be configured to communicate according to a different wireless modality, such as various versions of Bluetooth, near-field communications (NEC), ultra-high frequency (UHF), and so forth. Each wireless antenna(s) 36 may be configured to communicate in one band or across multiple bands. The wireless controller 32 is configured to communicate according to the various wireless modalities corresponding to the one or more antennas 36. The wireless controller 32 may be a unitary device or may be multiple individual controllers each separately configured to communicate according to a different modality supported by the various antennas. In a non-limiting example, the wireless communication block 34 includes a single antenna 36 configured to communicate, sensor data to an external device, such as the wearable article processing system 1808 of FIG. 18, and/or the user device 1802 of FIG. 18, according to a Bluetooth standard or any other standard.

The wireless communication block 34 is configured to communicate via the various modalities with one or more external devices which are not themselves part of the wearable article. The external devices may be mobile devices, such as mobile phones (e.g., user device 2802), smartphones, personal digital assistants (PDAs), mobile music or media players, and so forth. The external device additionally or alternatively may be stationary or generally stationary, such as a race tracker or base station. The wireless communication block 34 may pair with a given external device according to conventional pairing mechanisms related to the given external device so as to establish a communication link between the wearable article and the external device.

In various examples, the control block 40 includes as a separate component or implements with the processor(s) 42 and/or the input/output block 44 implements an analog-to-digital converter (ADC) and rate smoothing and/or filtering of the signals from the one or more sensors 48. In various examples, the ADC converts the input analog signal from approximately zero (0) Volts to approximately five (5) Volts to and eight-bit digital signal at a sampling rate of front approximately ten (10) Hertz to fifty (50) Hertz in an example, the sample rate is thirty (30) Hertz.

In various examples, the input/output block 44 and/or the processor(s) 42 utilizes a rolling weighted average of the digital output from the ADC for each sensor data value received from the sensor(s) 48. In an example, the processor(s) 42 applies a rolling weighted average of 0.2 for a current output from the ADC for an accelerometer sensor and 0.8 for the previous rolling weighted average of the output of the accelerometer sensor. Thus, the current rolling weighted average for the output from a given accelerometer sensor is eighty (80) percent based on the previous average and twenty (20) percent based on the current output from the ADC for that pressure sensor. It is noted and emphasized that each sensor may be assessed for its rolling weighted average separately and independently.

Looking now to FIG. 20, computing device 8 includes a bus 10 that directly or indirectly couples the following devices: memory 12, one or more processors 14, one or more presentation components 16, input/output (I/O) ports 18, input/output components 20, and an illustrative power supply 22. Bus 10 represents what may be one or more busses (such as an address bus, data bus, or combination thereof). Although the various blocks of FIG. 20 are shown with lines for the sake of clarity, in reality, delineating various components is not so clear, and metaphorically, the lines would more accurately be grey and fuzzy. For example, one may consider a presentation component such as a display device to be an I/O component. Also, processors have memory. The inventor recognizes that such is the nature of the art, and reiterates that the diagram of FIG. 20 is merely illustrative of an exemplary computing device that can be used in connection with one or more aspects of the present invention. Distinction is not made between such categories as “workstation,” “server,” “laptop,” “hand-held device,” etc., as all are contemplated within the scope of FIG. 20 and reference to “computing device.”

Computing device 8 typically includes a variety of computer-readable media. Computer-readable media can be any available media that can be accessed by computing device 8 and includes both volatile and nonvolatile media, and removable and non-removable media. By way of example, and not limitation, computer-readable media may comprise computer storage media and communication media. Computer storage media includes both volatile and nonvolatile, removable and non-removable media implemented in any method or technology for storage of information such as computer-readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can be accessed by computing device 8. Computer storage media does not comprise signals per se. Communication media typically embodies computer-readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media. In various aspects, the computing device 8 represents the physical architecture of the user device 1802, user device 912 of FIG. 9, or the wearable article processing system 1808 of FIG. 18.

Memory 12 includes computer-storage media in the form of volatile and/or nonvolatile memory. The memory may be removable, non-removable, or a combination thereof. Exemplary hardware devices include solid-state memory, hard drives, optical-disc drives, etc. Computing device 8 includes one or more processors that read data from various entities such as memory 12 or I/O components 20. Presentation component(s) 16 present data indications to a user or other device. Exemplary presentation components include a display device, speaker, printing component, vibrating component, etc. In some aspects, the memory includes program instructions that, when executed by one or more processors, cause the one or more processors to perform any operations described herein, such as the processes 1100, 1200, 1300, 1400, 1500, and/or 1600 of FIGS. 11, 12, 13, 14, 15, and/or 16 or any functionality described herein.

I/O ports 18 allow computing device 8 to be logically coupled to other devices including I/O components 20, some of which may be built in. Illustrative components include a microphone, joystick, game pad, satellite dish, scanner, printer, wireless device, etc. The I/O components 20 may provide a natural user interface (NUI) that processes air gestures, voice, or other physiological inputs generated by a user. In some instances, inputs may be transmitted to an appropriate network element for further processing. An NUI may implement any combination of speech recognition, stylus recognition, facial recognition, biometric recognition, gesture recognition both on screen and adjacent to the screen, air gestures, head and eye tracking, and touch recognition (as described in more detail below) associated with a display of the computing device 8. The computing device 8 may be equipped with depth cameras, such as stereoscopic camera systems, infrared camera systems, RGB camera systems, touchscreen technology, and combinations of these, for gesture detection and recognition. Additionally, the computing device 8 may be equipped with accelerometers or gyroscopes that enable detection of motion. The output of the accelerometers or gyroscopes may be provided to the display of the computing device 8 to render immersive augmented reality or virtual reality.

As can be understood, aspects of the present invention provide for, among other things, generating proof and attestation service notifications corresponding to a determined veracity of a claim. The present invention has been described in relation to particular aspects, which are intended in all respects to be illustrative rather than restrictive. Alternative aspects will become apparent to those of ordinary skill in the art to which the present invention pertains without departing from its scope.

From the foregoing, it will be seen that this invention is one well adapted to attain all the ends and objects set forth above, together with other advantages which are obvious and inherent to the system and method. It will be understood that certain features and sub combinations are of utility and may be employed without reference to other features and sub combinations. This is contemplated by and is within the scope of the claims.

The subject matter of the present invention is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” may be used herein to connote different elements of methods employed, the terms should not be interpreted as implying any particular order among or between various steps herein disclosed unless and except when the order of individual steps is explicitly described.

The following clauses represent example aspects of concepts contemplated herein. Any one of the following clauses may be combined in a multiple dependent manner to depend from one or more other clauses. Further, any combination of dependent clauses (clauses that explicitly depend from a previous clause) may be combined while staying within the scope of aspects contemplated herein. The following clauses are examples and are not limiting.

Clause 1: A computer-implemented method comprising, receiving, at a first time, first sensor data via one or more sensors included in an article of footwear, the one or more sensors excluding a force sensor; receiving, at a second time subsequent to the first time, second sensor data via the one or more sensors; based at least in part on the first sensor data and the second sensor data, estimating a change in force representative of at least a partial force curve; and at least partially in response to the estimating, sending, over a computer network and to a user device for presentation, at least one of: the received first sensor data, the received second sensor data, or a visual associated with the estimated change in force.

Clause 2: The computer-implemented method of clause 1, wherein the estimating is based on using a Convolutional Neural Network (CNN).

Clause 3: The computer-implemented method of any of clauses 1-2, wherein a first portion of the CNN is stored to the article of footwear, and wherein the first portion includes an intermediate layer that produces an output indicative of the estimation, and wherein the first portion does not include a dense layer.

Clause 4: The computer-implemented method of any of clauses 1-3, further comprising: adding one or more run features to one of, a first input into a convolutional layer at the CNN or a second input to a flattening layer at the CNN, wherein the estimating is based at least in part on the adding of the one or more run features.

Clause 5: The computer-implemented method of any of clauses 1-4, wherein the one or more sensors are included in an Inertial Measurement Unit (IMU) that includes: an accelerometer that provides a first portion of the sensor data along a longitudinal axis and a vertical axis, and wherein the estimation is not based on the accelerometer providing the first portion along a mediolateral axis; and a gyroscope that provides a second portion of the sensor data via a pitch axis, and wherein the estimation is not based on the gyroscope providing the second portion via a yaw axis or a roll axis.

Clause 6: The computer-implemented method of any of clauses 1-5, wherein the estimating is based on processing only a fixed window of sensor data indicative of a gait phase of a full gait cycle, and wherein other sensor data indicative of other portions of the full gait cycle are not processed for the estimating.

Clause 7: The computer-implemented method of any of clauses 1-6, wherein the visual includes at least one of: a ground force curve, an immersive technology element, a gaming element of a video game, or a user interface element that warns a user when the force exceeds a threshold.

Clause 8: The computer-implemented method of any of clauses 1-7, wherein the method further comprising: determining that at least one of, the first sensor data, the second sensor data, or the force exceeds a threshold; in response to the determining that at least one of, the first sensor data, the second sensor data, or the force exceeds the threshold, tracking at least one of, the first sensor data, the second sensor data, or the force for a certain time period; and in response to the tracking of at least one of, the first sensor data, the second sensor data, or the force for the certain time period, sending a notification to the user device that indicates at least one of, the tracked first sensor data, the tracked second sensor data, or the tracked force has exceeded the threshold.

Clause 9: The computer-implemented method of any of clauses 1-8, further comprising, based at least in part on estimating of the change in force, causing the article of footwear to apply a resist or assist force.

Clause 10: The computer-implemented method of any of clauses 1-9, further comprising, based at least in part on estimating of the change in force, classifying a lower limb gesture, and wherein the lower limb gesture includes at least one of: a stomp, a kick, or a jump.

Clause 11: The computer-implemented method of any of clauses 1-10, further comprising, based at least in part on one of the first sensor data or the second sensor data, classifying an activity type that a wearer of the article of footwear has engaged in, and wherein the estimation is based at least in part on the classification of the activity type, and wherein the activity type includes at least one of, running, walking, jogging, jumping, landing, or a gait phase of a gait cycle.

Clause 12: The computer-implemented method of any of clauses 1-11, wherein the classification is based on using a piecewise model.

Clause 13: The computer-implemented method of any of clauses 1-12, further comprising, based at least in part on one of the first sensor data or the second sensor data, estimating one or more gait metrics, and wherein the estimation of the change in force is further based on the estimation of the one or more gait metrics, and wherein the one or more gait metrics include at least one of, a quantity of strides a wearer of the article of footwear makes, an activity type of the wearer, a speed of the wearer, a Gaussian Curvature Tensor value, a stride length, a quantity of steps per minute, a foot strike angle, a Vertical Ground Reaction Force (VGRF) average, a VGRF active peak, a VGRF impulse, or a VGRF instantaneous Loading Rate.

Clause 14: The computer-implemented method of any of clauses 1-13, further comprising estimating and scaling a shape of the force curve based at least in part on the estimating of the change in force.

Clause 15: An article of footwear configured to conform to a human foot, the article of footwear comprising: one or more sensors; and one or more processors communicatively coupled to the one or more sensors, wherein the one or more processors are configured to perform operations comprising: receiving, at a first time, first sensor data from one or more sensors; receiving, at a second time subsequent to the first time, second sensor data from the one or more sensors; based at least in part on the first sensor data and the second sensor data, estimating a change in force representative of at least a partial force curve; and at least partially in response to the estimating, sending, over a computer network and to a user device for presentation, at least one of: the received first sensor data, the received second sensor data, or a visual associated with the estimated change in force.

Clause 16: The article of footwear of clause 15, wherein the estimating is based on using a Convolutional Neural Network (CNN) stored to a computer memory within the article of footwear.

Clause 17: The article of footwear of clauses 15-16, wherein a first portion of the CNN is stored to the computer memory, and wherein the first portion includes an intermediate layer that produces an output indicative of the estimation, and wherein the first portion does not include a dense layer, and wherein a second portion of the CNN is stored to the user device, and wherein the second portion includes the dense layer.

Clause 18: The article of footwear of clauses 15-17, wherein the one or more processors are configured to perform further operations comprising: adding one or more run features to one of, a first input into a convolutional layer at the CNN or a second input to a flattening layer at the CNN, wherein the estimating is based at least in part on the adding of the one or more run features.

Clause 19: The article of footwear of clauses 15-18, wherein the one or more sensors are included in an Inertial Measurement Unit (IMU) that includes: an accelerometer that provides a first portion of the sensor data along a longitudinal axis and a vertical axis, and wherein the estimation is not based on the accelerometer providing the first portion along a mediolateral axis; and a gyroscope that provides a second portion of the sensor data via a pitch axis, and wherein the estimation is not based on the gyroscope providing the second portion via a yaw axis or a roll axis.

Clause 20: The article of footwear of clauses 15-19, wherein the estimating is based on the one or more processors processing only a fixed window of sensor data indicative of a gait phase of a full gait cycle, and wherein other sensor data indicative of other portions of the full gait cycle are not processed for the estimating.

Clause 21: The article of footwear of clauses 15-20, wherein the visual includes at least one of: a ground force curve, an immersive technology element, a gaming element of a video game, or a user interface element that warns a user when the force exceeds a threshold.

Clause 22: The article of footwear of clauses 15-21, wherein the one or more processors are configured to perform further operations comprising: determining that at least one of, the first sensor data, the second sensor data, or the force exceeds a threshold; and based on the determining that at least one of the first sensor data, the second sensor data, or the force exceeds the threshold, sending a notification to the user device.

Clause 23: The article of footwear of clauses 15-22, wherein the one or more processors are configured to perform further operations comprising, based at least in part on estimating of the change in force, causing the article of footwear to apply a resist or assist force.

Clause 24: The article of footwear of clauses 15-23, wherein the one or more processors are configured to perform further operations comprising, based at least in part on estimating of the change in force, classifying a lower limb gesture, and wherein the lower limb gesture includes at least one of: a stomp, a kick, or a jump.

Clause 25: The article of footwear of clauses 15-24, wherein the one or more processors are configured to perform further operations comprising, based at least in part on one of the first sensor data or the second sensor data, classifying an activity type that a wearer of the article of footwear has engaged in, and wherein the estimation is based at least in part on the classification of the activity type, and wherein the activity type includes at least one of, running, walking, jogging, jumping, landing, or a gait phase of a gait cycle, and wherein the classification is based on using a piecewise model.

Clause 26: The article of footwear of clauses 15-25, wherein the one or more processors are configured to perform further operations comprising, based at least in part on one of the first sensor data or the second sensor data, estimating one or more gait metrics, and wherein the estimation of the change in force is further based on the estimation of the one or more gait metrics, and wherein the one or more gait metrics include at least one of, a quantity of strides a wearer of the article of footwear makes, an activity type of the wearer, a speed of the wearer, a Gaussian Curvature Tensor value, a stride length, a quantity of steps per minute, a foot strike angle, a Vertical Ground Reaction Force (VGRF) average, a VGRF active peak, a VGRF impulse, or a VGRF instantaneous Loading Rate.

Clause 27: The article of footwear of clauses 15-26, wherein the one or more processors are configured to perform further operations comprising estimating and scaling a shape of the force curve based at least in part on the estimating of the change in force.

Clause 28. A system comprising: a wearable article comprising one or more sensors; and one or more processors communicatively coupled to the one or more sensors, wherein the one or more processors are configured to perform operations comprising: receiving, at a first time, first sensor data from one or more sensors; receiving, at a second time subsequent to the first time, second sensor data from the one or more sensors; based at least in part on the first sensor data and the second sensor data, estimating a change in force representative of at least a partial force curve; and at least partially in response to the estimating, causing presentation, at a user device, at least one of: the received first sensor data, the received second sensor data, or a visual associated with the estimated change in force.

Clause 29. A computer-implemented method comprising: receiving, at a first time, a set of sensor data via one or more sensors included in an article of footwear, the one or more sensors excluding a force sensor; based at least in part on the received set of sensor data, estimating one or more gait metrics; and based at least in part on the estimating, engaging in at least one of: providing the estimated gait metrics to a model for further processing, or causing presentation, at a user device, of a visual element.

Clause 30: The computer-method of clause 29, further comprising, in response to providing the estimated gait metrics to the model, estimating, via the model, a change in force representative of at least a partial force curve.

Clause 31: The computer-implemented method of clauses 29-30, wherein a first portion of the model is stored to the article of footwear, and wherein the first portion includes an intermediate layer that produces an output indicative of the estimation of the change in force, and wherein the first portion does not include a dense layer, and wherein a second portion of the model is stored to the user device, and wherein the second portion includes the dense layer.

Clause 32: The computer-implemented method of clauses 29-31, further comprising: adding the one or more gait metrics to one of, a first input into a convolutional layer at a Convolutional Neural Network (CNN) or a second input to a flattening layer at the CNN, wherein an estimating of a change in force is based at least in part on the adding of the one or more gait metrics.

Clause 33: The computer-implemented method of clauses 29-32, wherein the estimating is based on processing only a fixed window of sensor data indicative of a gait phase of a full gait cycle, and wherein other sensor data indicative of other portions of the full gait cycle are not processed for the estimating.

Clause 34: The computer-implemented method of clauses 29-33, wherein the visual element includes at least one of: a ground force curve, an immersive technology element, a gaming element of a video game, or a user interface element.

Clause 35: The computer-implemented method of clauses 29-34, further comprising, based at least in part on estimating, causing the article of footwear to apply a resist or assist force.

Clause 36: An article of footwear comprising: one or more sensors; and one or more processors communicatively coupled to the one or more sensors, wherein the one or more processors are configured to perform operations comprising: receiving a set of sensor data from the one or more sensors; based at least in part on the received set of sensor data, estimating an activity type of a wearer associated with the article of footwear; and based at least in part on the estimating, engaging in at least one of, providing the estimated activity type to a model for further processing or causing presentation, to a user device, of a visual element.

Clause 37: The article of footwear of clause 36, wherein the one or more processor are configured to perform further operations comprising, in response to providing the estimated activity type to the model, estimating, via the model, a change in force representative of at least a partial force curve.

Clause 38: The article of footwear of clauses 36-37, wherein a first portion of the model is stored to the article of footwear, and wherein the first portion includes an intermediate layer that produces an output indicative of the estimation of the change in force, and wherein the first portion does not include a dense layer, and wherein a second portion of the model is stored to the user device, and wherein the second portion includes the dense layer.

Clause 39: The article of footwear of clauses 36-38, wherein the estimating is based on processing only a fixed window of sensor data indicative of a gait phase of a full gait cycle, and wherein other sensor data indicative of other portions of the full gait cycle are not processed for the estimating.

Clause 40: The article of footwear of clauses 36-39, wherein the visual element includes at least one of: a force curve, an immersive technology element, a gaming element of a video game, or a user interface element.

Clause 41: The article of footwear of clauses 36-40, further comprising, based at least in part on estimating, causing the article of footwear to apply a resist or assist force.

Clause 42: A computer-implemented method comprising: receiving sensor data over a first time period and via one or more sensors included in an article of footwear, the one or more sensors excluding a force sensor; based at least in part on analyzing the sensor data, estimating a quantity of force; determining that the estimated quantity of force exceeds a threshold;

at least partially in response to the determining that the estimated quantity of force exceeds the threshold, sending, over a computer network and to a user device for presentation, at least one of: the received sensor data, or a visual associated with the estimated quantity of force.

The present technology has been described in relation to particular aspects, which are intended in all respects to be illustrative rather than restrictive. Alternative aspects will become apparent to those of ordinary skill in the art to which the present technology pertains without departing from its scope.

Having identified various components utilized herein, it should be understood that any number of components and arrangements can be employed to achieve the desired functionality within the scope of the present disclosure. For example, the components in the aspects depicted in the figures are shown with lines for the sake of conceptual clarity. Other arrangements of these and other components can also be implemented. For example, although some components are depicted as single components, many of the elements described herein can be implemented as discrete or distributed components or in conjunction with other components, and in any suitable combination and location. Some elements can be omitted altogether. Moreover, various functions described herein as being performed by one or more entities can be carried out by hardware, firmware, and/or software, as described below. For instance, various functions can be carried out by a processor executing instructions stored in memory. As such, other arrangements and elements (e.g., machines, interfaces, functions, orders, and groupings of functions) can be used in addition to or instead of those shown.

Aspects described herein can be combined with one or more of the specifically described alternatives. In particular, an aspect that is claimed can contain a reference, in the alternative, to more than one other aspect. The aspect that is claimed can specify a further limitation of the subject matter claimed.

The subject matter of aspects of the technology is described with specificity herein to meet statutory requirements. However, the description itself is not intended to limit the scope of this patent. Rather, the inventors have contemplated that the claimed subject matter might also be embodied in other ways, to include different steps or combinations of steps similar to the ones described in this document, in conjunction with other present or future technologies. Moreover, although the terms “step” and/or “block” can be used herein to connote different elements of methods employed, the terms should not be interpreted as implying any particular order among or between various steps herein disclosed unless and except when the order of individual steps is explicitly described.

For purposes of this disclosure, the word “including” has the same broad meaning as the word “comprising,” and the word “accessing” comprises “receiving,” “referencing,” or “retrieving.” Further, the word “communicating” has the same broad meaning as the word “receiving,” or “transmitting” facilitated by software or hardware-based buses, receivers, or transmitters using communication media described herein. In addition, words such as “a” and “an,” unless otherwise indicated to the contrary, include the plural as well as the singular. Thus, for example, the constraint of “a feature” is satisfied where one or more features are present. Also, the term “or” includes the conjunctive, the disjunctive, and both (a or b thus includes either a or b, as well as a and b).

For purposes of a detailed discussion above, aspects of the present technology are described with reference to a distributed computing environment; however, the distributed computing environment depicted herein is merely exemplary. Components can be configured for performing novel aspects of aspects, where the term “configured for” can refer to “programmed to” perform particular tasks or implement particular abstract data types using code. Further, while aspects of the present technology can generally refer to the technical solution environment and the schematics described herein, it is understood that the techniques described can be extended to other implementation contexts.

From the foregoing, it will be seen that this technology is one well adapted to attain all the ends and objects set forth above, together with other advantages which are obvious and inherent to the system and method. It will be understood that certain features and subcombinations are of utility and can be employed without reference to other features and subcombinations. This is contemplated by and is within the scope of the claims.

What is claimed is:

1. A computer-implemented method comprising:
receiving, at a first time, first sensor data via one or more sensors included in an article of footwear, the one or more sensors excluding a force sensor;
receiving, at a second time subsequent to the first time, second sensor data via the one or more sensors;
based at least in part on the first sensor data and the second sensor data, estimating a change in force representative of at least a partial force curve; and
at least partially in response to the estimating, sending, over a computer network and to a user device for presentation, at least one of: the received first sensor data, the received second sensor data, or a visual associated with the estimated change in force.

2. The method of claim 1, wherein the estimating is based on using a Convolutional Neural Network (CNN).

3. The method of claim 2, wherein a first portion of the CNN is stored to the article of footwear, and wherein the first portion includes an intermediate layer that produces an output indicative of the estimation of the change in force, and wherein the first portion does not include a dense layer.

4. The method of claim 2, further comprising: adding one or more run features to one of, a first input into a convolutional layer at the CNN or a second input to a flattening layer at the CNN, wherein the estimation of the change in force is based at least in part on the adding of the one or more run features.

5. The method of claim 1, wherein the one or more sensors are included in an Inertial Measurement Unit (IMU) that includes:
an accelerometer that provides a first portion of the first sensor data along a longitudinal axis and a vertical axis, and wherein the estimation of the change in force is not based on the accelerometer providing the first portion along a mediolateral axis; and
a gyroscope that provides a second portion of the first sensor data via a pitch axis, and wherein the estimation of the change in force is not based on the gyroscope providing the second portion via a yaw axis or a roll axis.

6. The method of claim 1, wherein the estimating is based on processing only a fixed window of sensor data indicative of a gait phase of a full gait cycle, and wherein other sensor data indicative of other portions of the full gait cycle are not processed for the estimating.

7. The method of claim 1, wherein the visual includes at least one of: a ground force curve, an immersive technology element, a gaming element of a video game, or a user interface element that warns a user when the force exceeds a threshold.

8. The method of claim 1, wherein the method further comprising:
determining that at least one of, the first sensor data, the second sensor data, or the force exceeds a threshold; and
in response to the determining that at least one of, the first sensor data, the second sensor data, or the force exceeds the threshold, tracking at least one of, the first sensor data, the second sensor data, or the force for a certain time period; and in response to the tracking of at least one of, the first sensor data, the second sensor data, or the force for the certain time period, sending a notification to the user device that indicates at least one of, the tracked first sensor data, the tracked second sensor data, or the tracked force has exceeded the threshold.

9. The method of claim 1, further comprising,
based at least in part on estimating of the change in force, causing the article of footwear to apply a resist or assist force.

10. The method of claim 1, further comprising,
based at least in part on estimating of the change in force, classifying a lower limb gesture, and wherein the lower limb gesture includes at least one of: a stomp, a kick, or a jump.

11. The method of claim 1, further comprising,
based at least in part on one of the first sensor data or the second sensor data, classifying an activity type that a wearer of the article of footwear has engaged in, and wherein the estimation of the change in force is based at least in part on the classification of the activity type, and wherein the activity type includes at least one of, running, walking, jogging, jumping, landing, or a gait phase of a gait cycle.

12. The method of claim 11, wherein the classification is based on using a piecewise model.

13. The method of claim 1, further comprising,
based at least in part on one of the first sensor data or the second sensor data, estimating one or more gait metrics, and wherein the estimation of the change in force is further based on the estimation of the one or more gait metrics, and wherein the one or more gait metrics include at least one of, a quantity of strides a wearer of the article of footwear makes, an activity type of the wearer, a speed of the wearer, a Gaussian Curvature Tensor value, a stride length, a quantity of steps per minute, a foot strike angle, a Vertical Ground Reaction Force (VGRF) average, a VGRF active peak, a VGRF impulse, or a VGRF instantaneous Loading Rate.

14. The method of claim 1, further comprising estimating and scaling a shape of the force curve based at least in part on the estimating of the change in force.

15. An article of footwear configured to conform to a human foot, the article of footwear comprising:
one or more sensors; and
one or more processors communicatively coupled to the one or more sensors, wherein the one or more processors are configured to perform operations comprising:
receiving, at a first time, first sensor data from the one or more sensors;
receiving, at a second time subsequent to the first time, second sensor data from the one or more sensors;
based at least in part on the first sensor data and the second sensor data, estimating a change in force representative of at least a partial force curve; and
at least partially in response to the estimating of the change in force, sending, over a computer network and to a user device for presentation, at least one of: the received first sensor data, the received second sensor data, or a visual associated with the estimated change in force.

16. The article of footwear of claim 15, wherein the estimating is based on using a Convolutional Neural Network (CNN) stored to a computer memory within the article of footwear.

17. The article of footwear of claim 15, wherein a first portion of the CNN is stored to computer memory, and wherein the first portion includes an intermediate layer that produces an output indicative of the estimating of the change in force, and wherein the first portion does not include a dense layer, and wherein a second portion of the CNN is stored to the user device, and wherein the second portion includes the dense layer.

18. The article of footwear of claim 15, wherein the one or more processors are configured to perform further operations comprising: adding one or more run features to one of, a first input into a convolutional layer at the CNN or a second input to a flattening layer at the CNN, wherein the estimating of the change in force is based at least in part on the adding of the one or more run features.

19. The article of footwear of claim 15, wherein the one or more sensors are included in an Inertial Measurement Unit (IMU) that includes:
an accelerometer that provides a first portion of the first sensor data along a longitudinal axis and a vertical axis, and wherein the estimation of the change in force is not based on the accelerometer providing the first portion along a mediolateral axis; and
a gyroscope that provides a second portion of the first sensor data via a pitch axis, and wherein the estimation of the change in force is not based on the gyroscope providing the second portion via a yaw axis or a roll axis.

20. The article of footwear of claim 15, wherein the estimating is based on the one or more processors processing only a fixed window of sensor data indicative of a gait phase of a full gait cycle, and wherein other sensor data indicative of other portions of the full gait cycle are not processed for the estimating.

21. The article of footwear of claim 15, wherein the visual includes at least one of: a ground force curve, an immersive technology element, a gaming element of a video game, or a user interface element that warns a user when the force exceeds a threshold.

22. The article of footwear of claim 15, wherein the one or more processors are configured to perform further operations comprising:
determining that at least one of, the first sensor data, the second sensor data, or the force exceeds a threshold; and
based on the determining that at least one of the first sensor data, the second sensor data, or the force exceeds the threshold, sending a notification to the user device.

23. The article of footwear of claim 15, wherein the one or more processors are configured to perform further operations comprising,
based at least in part on estimating of the change in force, causing the article of footwear to apply a resist or assist force.

24. The article of footwear of claim 15, wherein the one or more processors are configured to perform further operations comprising,
based at least in part on estimating of the change in force, classifying a lower limb gesture, and wherein the lower limb gesture includes at least one of: a stomp, a kick, or a jump.

25. The article of footwear claim 15, wherein the one or more processors are configured to perform further operations comprising,
based at least in part on one of the first sensor data or the second sensor data, classifying an activity type that a wearer of the article of footwear has engaged in, and wherein the estimation of the change in force is based at least in part on the classification of the activity type, and wherein the activity type includes at least one of, running, walking, jogging, jumping, landing, or a gait phase of a gait cycle, and wherein the classification is based on using a piecewise model.

26. The article of footwear of claim 15, wherein the one or more processors are configured to perform further operations comprising,
based at least in part on one of the first sensor data or the second sensor data, estimating one or more gait metrics, and wherein the estimation of the change in force is further based on the estimation of the one or more gait metrics, and wherein the one or more gait metrics include at least one of, a quantity of strides a wearer of the article of footwear makes, an activity type of the wearer, a speed of the wearer, a Gaussian Curvature Tensor value, a stride length, a quantity of steps per minute, a foot strike angle, a Vertical Ground Reaction Force (VGRF) average, a VGRF active peak, a VGRF impulse, or a VGRF instantaneous Loading Rate.

27. The article of footwear of claim 15, wherein the one or more processors are configured to perform further operations comprising estimating and scaling a shape of the force curve based at least in part on the estimating of the change in force.

28. A system comprising:
a wearable article comprising one or more sensors; and
one or more processors communicatively coupled to the one or more sensors, wherein the one or more processors are configured to perform operations comprising:
receiving, at a first time, first sensor data from the one or more sensors;
receiving, at a second time subsequent to the first time, second sensor data from the one or more sensors;
based at least in part on the first sensor data and the second sensor data, estimating a change in force representative of at least a partial force curve; and
at least partially in response to the estimating, causing presentation, at a user device, at least one of: the received first sensor data, the received second sensor data, or a visual associated with the estimated change in force.

29. A computer-implemented method comprising:
receiving, at a first time, a set of sensor data via one or more sensors included in an article of footwear, the one or more sensors excluding a force sensor;
based at least in part on the received set of sensor data, estimating one or more gait metrics; and
based at least in part on the estimating, engaging in at least one of: providing the estimated one or more gait metrics to a model for further processing, or causing presentation, at a user device, of a visual element.

30. The method of claim 29, further comprising, in response to providing the estimated one or more gait metrics to the model, estimating, via the model, a change in force representative of at least a partial force curve.

31. The method of claim 29, wherein a first portion of the model is stored to the article of footwear, and wherein the first portion includes an intermediate layer that produces an output indicative of the estimation of the one or more gait metrics, and wherein the first portion does not include a dense layer, and wherein a second portion of the model is stored to the user device, and wherein the second portion includes the dense layer.

32. The method of claim 29, further comprising: adding the one or more gait metrics to one of, a first input into a convolutional layer at a Convolutional Neural Network (CNN) or a second input to a flattening layer at the CNN, wherein an estimating of a change in force is based at least in part on the adding of the one or more gait metrics.

33. The method of claim 29, wherein the estimating is based on processing only a fixed window of sensor data indicative of a gait phase of a full gait cycle, and wherein other sensor data indicative of other portions of the full gait cycle are not processed for the estimating.

34. The method of claim 29, wherein the visual element includes at least one of: a ground force curve, an immersive technology element, a gaming element of a video game, or a user interface element.

35. The method of claim 29, further comprising,
based at least in part on estimating, causing the article of footwear to apply a resist or assist force.

36. An article of footwear comprising:
one or more sensors; and
one or more processors communicatively coupled to the one or more sensors, wherein the one or more processors are configured to perform operations comprising:
receiving a set of sensor data from the one or more sensors;
based at least in part on the received set of sensor data, estimating an activity type of a wearer associated with the article of footwear; and
based at least in part on the estimating, engaging in at least one of, providing the estimated activity type to a model for further processing or causing presentation, to a user device, of a visual element.

37. The article of footwear of claim 36, wherein the one or more processors are configured to perform further operations comprising, in response to providing the estimated activity type to the model, estimating, via the model, a change in force representative of at least a partial force curve.

38. The article of footwear of claim 37, wherein a first portion of the model is stored to the article of footwear, and wherein the first portion includes an intermediate layer that produces an output indicative of the estimation of the change in force, and wherein the first portion does not include a dense layer, and wherein a second portion of the model is stored to the user device, and wherein the second portion includes the dense layer.

39. The article of footwear of claim 36, wherein the estimating is based on processing only a fixed window of sensor data indicative of a gait phase of a full gait cycle, and wherein other sensor data indicative of other portions of the full gait cycle are not processed for the estimating.

40. The article of footwear of claim 36, wherein the visual element includes at least one of: a force curve, an immersive technology element, a gaming element of a video game, or a user interface element.

41. The article of footwear of claim 36, further comprising,
based at least in part on estimating, causing the article of footwear to apply a resist or assist force.

42. A computer-implemented method comprising:
receiving sensor data over a first time period and via one or more sensors included in an article of footwear, the one or more sensors excluding a force sensor;
based at least in part on analyzing the sensor data, estimating a quantity of force;
determining that the estimated quantity of force exceeds a threshold; and
at least partially in response to the determining that the estimated quantity of force exceeds the threshold, sending, over a computer network and to a user device for presentation, at least one of: the received sensor data, or a visual associated with the estimated quantity of force.
